(19) United States
(12) Patent Application Publication
(10) Pub. No.: US 2015/0168419 A1
(43) Pub. Date: Jun. 18, 2015

(54) PREDICTION OF A SMALL-FOR-GESTATIONAL AGE (SGA) INFANT

(71) Applicant: UNIVERSITY COLLEGE CORK - NATIONAL UNIVERSITY OF IRELAND, CORK, Cork (IE)

(72) Inventors: Louise Kenny, Co. Cork (IE); Philip Baker, Auckland (NZ)

(73) Assignee: UNIVERSITY COLLEGE CORK - NATIONAL UNIVERSITY OF IRELAND, CORK, Cork (IE)

(21) Appl. No.: 14/550,058

(22) Filed: Nov. 21, 2014

Related U.S. Application Data
(63) Continuation of application No. 13/885,190, filed on Jul. 23, 2013, now abandoned.
(60) Provisional application No. 61/414,243, filed on Nov. 16, 2010.

(51) Int. Cl. G01N 33/68; G01N 33/92

(52) U.S. Cl. CPC G01N 33/689; G01N 33/92; G01N 2800/368

(57) ABSTRACT

A method of predicting a SGA infant in a patient at a pre-symptomatic gestational stage is described. The method comprises a step of assaying a biological sample from the patient for an abundance of a plurality of metabolite biomarkers selected from the 19 metabolite biomarkers of Table IV, correlating the abundance of the plurality of metabolite biomarkers with a metabolite fingerprint of SGA shown in Table IV, and predicting SGA based on the level of correlation between the abundance of the plurality of metabolite biomarkers and the metabolite fingerprint of Table IV.

### CROSS-REFERENCE TO RELATED APPLICATIONS

This application is a continuation of U.S. application Ser. No. 13/885,190 filed on Jul. 23, 2013, which is a 35 U.S.C. §371 National Phase Entry Application of International Application No. PCT/EP2011/070299 filed Nov. 16, 2011, which designates the U.S., and which claims benefit under 35 U.S.C. §119(e) of U.S. Provisional Application No. 61/414,243, filed Nov. 16, 2010, the contents of which are incorporated herein by reference in their entireties.

### FIELD OF THE INVENTION

The invention relates to a method of predicting a Small-for-gestational age (SGA) infant in a patient at a pre-symptomatic gestational stage.

### BACKGROUND OF THE INVENTION

Intrauterine growth restriction (IUGR), in which a baby fails to reach its growth potential, is a serious complication of pregnancy, complicating between 3-10% of all first time births. The Perinatal Mortality Rate (PNMR) in the IUGR fetus is four to ten times higher than that of normally grown infants (Chiswick M L, 1985) and approximately 5-10% of all pregnancies complicated by IUGR will result in either stillbirth or neonatal death (McIntire D D et al., 1990; Thornton J G et al, 2004). Suboptimal fetal growth is responsible for at least one quarter of all stillbirths (Thornton J G et al, 2004) and recent evidence suggests this figure is probably higher (Gardosi J et al. 2005) Analysis of over 23,000 fetal deaths in California on population-based percentile curves showed a strong link between low fetal weight for gestational age and fetal demise (Williams R L et al 1982). Immediate neonatal morbidity amongst survivors of IUGR pregnancies is also significantly increased. 25% to 40% of all neonates subjected to IUGR will require prolonged ventilation, and 4-6% will develop necrotizing enterocolitis (GRIT study group 2003). Similar data from the US demonstrates that 20% of all such surviving infants will develop respiratory distress syndrome requiring ventilation and 2% will develop sepsis (McIntire D D et al., 1990).

Of major public concern is the long-term paediatric morbidity for survivors of pregnancies affected by IUGR. At two years of age, 5% of survivors of pregnancies affected by IUGR will have cerebral palsy and this increases to 10% amongst survivors delivered at less than 30 weeks' gestation (Thornton J G et al, 2004). A further 1% of such survivors will have little or no vision and 8% of all growth restricted survivors will have some degree of neurological impairment. IUGR also has a significant public health impact in later adult life. It is now well established that a hostile intrauterine environment places surviving infants at significant risk of a variety of medical problems in adulthood. Adults who were growth restricted in utero have significantly increased risks of developing cardiovascular disease, including chronic hypertension, and also an increased risk of type II diabetes (Barker D J et al. 1993).

IUGR is a major public health problem as it is associated with fetal death, neonatal death, paediatric morbidity and cardiovascular disease in adulthood. Current screening strategies for IUGR are inadequate: twenty percent of pregnancies are considered antenatally to be “high risk”, the remainder being “low risk”. Examples of “high risk” pregnancies include stillbirth in a previous pregnancy, hypertensive complications of pregnancy, bleeding in pregnancy and rhesus disease. Once a pregnancy is identified as “high risk”, its outcome is maximised by sophisticated surveillance techniques and unexpected intrauterine death after viable gestation is reached in such pregnancies is now an uncommon event. Paradoxically, we have become so expert in looking after our “high-risk” patients that large studies in Dublin (Hospital NM Annual report 1991), Belfast (Sim D et al. 1993) and Nottingham {personal communication} have demonstrated that the perinatal mortality rate is now higher in the apparent “low-risk” pregnancy than the “high-risk” pregnancy. In the Nottingham study, which looked at 21,000 deliveries between 1995 and 1999, the PNMR in the high-risk population was two per thousand. This contrasts with a PNMR of eight per thousand in the low-risk population. Current assessment of fetal growth and the fetal environment is failing to predict and identify the high-risk fetus in the low risk population The National Institute for Clinical Excellence (NICE) suggests screening for fetal growth problems by performing maternal symphysiofundal height measurements using a tape measure (Woman. National Collaborating Centre for Women's and Children's Health, 2003) However, only 16% of small-for-gestational age (SGA) infants will be detected using current growth screening strategies in a low-risk population (Kean L et al. 1996). Moreover, at least 30% of SGA infants are healthy and constitutionally small and not IUGR. [McCowan L M, Harding J E, Stewart A W (2005) Customized birthweight centiles predict SGA pregnancies with perinatal morbidity. BJOG 112: 1026-1033.]

The fourth annual report of the Confidential Enquiry into Stillbirths and Deaths in Infancy in the United Kingdom (Dimond R 1997) found that the largest area identified for improvements in perinatal mortality was that of unexplained antepartum stillbirths; a large proportion of these ‘unexplained’ stillbirths are in pregnancies complicated by IUGR (Hovatta O et al. 1983; Ahlenius I et al. 1995). The development of a reliable and valid screening test for IUGR would enable women to be streamed according to obstetric risk. The intensity of antenatal care could then be matched to clinical need. The Euronatal audit study demonstrated that stillbirths might be reduced by an improvement in the detection and the management of severe IUGR (Richardus et al 1997). Identification and surveillance of IUGR fetuses in the “low-risk” population, with consequent improvement of the perinatal mortality to levels reported in “high-risk” populations would prevent 800 stillbirths in the UK each year, with further reductions in neonatal death, paediatric morbidity and cardiovascular disease in adulthood. Accurate risk assessment will enable recognised interventions to be focused on those likely to benefit; pregnant women will usually only take preventative therapy if they perceive a significant level of risk to themselves or their babies. The Cochrane analysis of aspirin trials reported a reduction in IUGR (Duley L et al. 2004) in high-risk pregnancies. Smoking cessation programmes have been shown to reduce low birth weight babies by 20% (Lumley J et al 2000). Equally, a test to confirm fetal wellbeing would reduce potentially harmful and expensive antenatal interventions such as unnecessary early induction of labour.

IUGR, its identification, management and complications are the major challenge facing the obstetrician of today. Current screening strategies are failing to predict and identify the high-risk fetus in the low risk population. Prevention of IUGR would have massive global health, economic and societal impact, whilst considerable economic benefit would accrue from streamlined healthcare.

Several biomarkers have been proposed for prediction of IUGR, including growth factors (Bhatia et al., 2002; Tjoa et al., 2003), placental hormones (Dugoff et al., 2004; Dugoff et al., 2005; Morris et al., 2008) and angiogenic factors (Bersinger & Odegard, 2004, 2005; Taylor et al., 2003). However, none (nor any combination) has shown satisfactory specificity and sensitivity to be clinically useful. Ultrasound is the cornerstone of antenatal diagnosis of IUGR, but this requires the clinician to suspect a baby is at risk of being SGA and organise a scan. Consequently, IUGR remains undiagnosed before birth in 40%-80% of cases, contributing to the high rate of stillbirth (Chang et al., 1992; Gardosi & Francis, 1999; Hall et al., 1980). At present there is no accurate way of predicting who is at risk of an IUGR baby amongst nulliparous women. Reliable antenatal identification of the IUGR fetus would facilitate appropriate surveillance, a greater understanding of the aetiological pathways and potentially reduce morbidity and perinatal death in this vulnerable group.

It is, therefore, desirable to provide compounds, compositions and/methods for the determination of complications of pregnancy, such as IUGR and/or SGA.

This background information is provided for the purpose of making known information believed by the applicant to be of possible relevance to the present invention. No admission is necessarily intended, nor should it be construed, that any of the preceding information constitutes prior art against the present invention.

### SUMMARY OF THE INVENTION

According to the invention, there is provided a method of predicting a SGA infact, particularly a SGA associated with IUGR infact, in a patient at a pre-symptomatic gestational stage comprising a step of assaying a biological sample obtained from the patient at a pre-symptomatic gestational stage for abundance of a plurality of metabolite biomarkers selected from the 19 metabolite biomarkers of Table IV, correlating the abundance of the plurality of metabolite biomarkers with a metabolite fingerprint of SGA shown in Table IV, and predicting SGA based on the level of correlation between the abundance of the plurality of metabolite biomarkers and the metabolite fingerprint of Table IV.

Thus, for each of the 19 biomarkers of Table IV, there is provided an indication of whether the biomarker is increased (up) or decreased (down) in a patient having SGA compared with a control. This enables an assay in which a plurality of metabolite biomarkers of Table IV (for example at least 2, 3, 4, 5, 6, 7, 8, 9, 10, 11, 12, 13, 14, 15, 16, 17, 18, or 19) from a patient are assayed for modulated abundance compared with a control, wherein SGA is predicted when modulated abundance of at least two biomarkers according to the metabolite fingerprint of Table IV is detected.

Suitably, the SGA (small-for-gestational age) condition includes FGR (fetal growth restriction) and IUGR (Intrauterine growth restriction).

Typically, the biological sample is selected from venous cord blood or maternal peripheral blood.

The term “pre-symptomatic gestational stage” means a stage of gestation where the symptoms of SGA are not yet apparent. Generally, this is prior to 20, 19, 18, or 17 weeks gestation. Typically, it refers to 15 weeks+/−4 weeks, 15 weeks+/−3 weeks, or 15 weeks+/−2 weeks. Thus, in a preferred embodiment of the invention, the biological sample is obtained from the patient at a pre-symptomatic, preferably at week 15 gestational stage+/−3 or 2 weeks.

In a preferred embodiment, the biological sample is assayed for substantially all of the 19 metabolite biomarkers of Table IV, and in which the levels of the assayed metabolite biomarkers are correlated with the metabolite fingerprint of SGA shown in Table IV, wherein SGA is predicted based on the level of correlation between the levels of the assayed metabolite biomarkers of Table IV and the metabolite fingerprint of Table IV. The term “substantially all of the 19 metabolite biomarkers” should be understood to mean at least 15, 16, 17 or 18 of the biomarkers of table IV.

In one embodiment, the biological sample is assayed for all of the 19 metabolite biomarkers of Table IV, and in which the levels of the assayed metabolite biomarkers are correlated with the metabolite fingerprint of SGA shown in Table IV, wherein SGA is predicted based on the level of correlation between the levels of the assayed metabolite biomarkers of Table IV and the metabolite fingerprint of Table IV.

Thus, in a preferred embodiment, the invention provides a method for predicting SGA in a patient at week 15 gestational stage+/−2 weeks comprising a step of assaying a venous cord blood or maternal peripheral blood sample from the patient for 19 metabolite biomarkers of Table IV, correlating the levels of the 19 assayed metabolite biomarkers with the metabolite fingerprint of pre-symptomatic SGA shown in Table IV, and predicting SGA based on the level of correlation between the assayed levels of the 19 metabolite biomarkers of Table IV and the metabolite fingerprint of Table IV.

In another aspect, the invention provides a system for performing a method of predicting SGA in a patient, the system comprising:
a determination system for detecting in a biological sample from the patient abundance of a plurality of metabolite biomarkers selected from Table IV;
optionally, a storage system for storing metabolite biomarker abundance data generated by the determination system;
a comparison system for comparing abundance data from the determination system with a metabolite finderprint of Table IV to provide a quantitative prediction of SGA; and
a display module for displaying the quantitative prediction of SGA.

Typically, the determination system comprises a mass spectrometer or liquid chromatograpy apparatus.

In one embodiment, the determination system is adapted for detecting in a biological sample from the patient abundance of substantially all, and ideally all, of the 19 metabolite biomarkers of Table IV.

Typically, the system of the invention is for performing a method of predicting SGA in a patient.

Ideally, the biological sample is venous cord blood or maternal peripheral blood, and is preferably obtained from the patient at 15 weeks gestation+/−3 or 2 weeks.

The 19 biomarkers of Table IV consist of the following:
1. phenylacetylglutamine or formyl-N-acetyl-5-methoxykynurenamine;
2. leucyl-leucyl-norleucine or sphingosine-1-phosphate;
3. cervonyl carnitine or 1α,25-dihydroxy-18-oxocholecalciferol;
4. (15Z)-Tetracosenoic acid or 10,13-Dimethyl-11-docosyne-10,13-diol or trans-selacholeic acid;
5. hexacosanedioic acid;
6. Pentacosenoic acid or Teasterone or Typhasterol;
7. Cycloheptanecarboxylic acid or cyclohexyl acetate or octenoic acid or methyl-heptenoic acid or 4-hydroxy-2-octenal or DL-2-aminooctanoic acid or 3-amino-octanoic acid;
8. Diglyceride(14:0/18:0) or Diglyceride(16:0/16:0);
9. Lyso-phosphocholine(18:2);
10. Hydroxybutyrate or hydroxyl-methylpropanoate or methyl methoxyacetate;
11. Lyso-phosphocholine and phosphocholine;
12. Phosphocholine;
13. Phosphocholine or ubiquinone;
14. Acetylleucyl-leucyl-norleucinal or oleoylglycerone phosphate or LPA(0:0/18:2(9Z,12Z)) or 1-16:1-lyso-prostaglandin E or phosphocholine(0-11:1(10E)/2:0) or (3s)-3,4-Di-N-hexanoyloxybutyl-1-phosphocholine or N-(3-hydroxy-propyl) arachidonoyl amine or N-(2-methoxy-ethyl) arachidonoyl amine or N-methyl N-(2-hydroxy-ethyl) arachidonoyl amine;
15. Lyso-phosphocholine (16:1) or cervonyl carnitine;
16. Sphinganine-1-phosphate;
17. Sphingosine-1-phosphate;
18. Pregnanediol-3-glucuronide or 3 alpha,20alpha-dyhydroxy-5beta-pregnane 3-glucuronide; and
19. 6-hydroxysphingosine or (4OH,8Z,t18:1) sphingosine or 15-methyl-15-PGD2 or 15R-PGE2 methyl ester.

In accordance with one aspect of the present invention there is provided a method of determining a metabolic fingerprint of SGA and/or IUGR comprising: obtaining a sample from said subject; measuring the metabolites within said sample and generating a metabolic profile of said sample; comparing the metabolic profile of said sample with a control metabolic profile, wherein said metabolic fingerprint is determined from the comparison of said sample and said control metabolic profiles.

In accordance with one aspect of the present invention, there is provided a method for determining SGA and/or IUGR in a subject, said method comprising analyzing a sample from said subject for a metabolic fingerprint of SGA and/or IUGR.

In accordance with one aspect of the present invention, there is provided a method comprising: obtaining a sample from a subject with, or suspected as having, SGA and/or IUGR; contacting the sample with a reagent to metabolite from the metabolic fingerprint for SGA and/or IUGR the reagent and the metabolite present in the sample; measuring the complex formed to determine an amount the metabolite in the sample, wherein the determination of SGA and/or IUGR is determined by the level of the metabolite in said sample.

In accordance with another aspect of the present invention, there is provided a method comprising: obtaining a biologic sample from a subject with, or suspected as having, SGA and/or IUGR; analyzing the sample using a machine wherein said machine having a detector set to detect metabolites within said complex to obtain metabolic fingerprint of the sample; determining SGA and/or IURG in said subject, wherein the determination of SGA and/or IURG in the subject is determined by the levels of the metabolites in said sample.

In another aspect of the present invention there is provided a kit for determining the metabolite profile in a biological sample, comprising regents to identify the metabolites of the metabolic profile and instructions for the use thereof.

In another aspect of the present invention there is provided a kit for determining a subject with SGA and/or IUGR comprising: instructions for determining a metabolic fingerprint in a biological sample from a subject; a reagent(s) for measuring the metabolic fingerprint in the biological sample from the subject, wherein the determination of SGA and/or IUGR in a subject indicated by the metabolite in the metabolic fingerprint in the sample.

### BRIEF DESCRIPTION OF THE DRAWINGS

Embodiments of the present invention will now be described, by way of example only, with reference to the attached Figures, wherein:

FIG. 1. A cross-validated PLS-DA model of all the venous cord plasma metabolite features detected was built using two latent factors. The resulting scores plot (la) presented as a scatter plot and box and whisker plot demonstrated clear differences between the SGA and control profiles with an R²=0.88, Q²=0.81, and an AUC of 1. The QC samples were not used in the model construction. These samples were simply projected through the model post-hoc. The relative lack of dispersion of the projected QC samples provided robust quality assurance of the model's precision. Permutation testing showed that the probability of a model of this quality randomly occurring was less than 0.001 (FIG. 6).

FIG. 2. A cross-validated PLS-DA model of all the RUPP plasma metabolite features detected was built using 2 latent factors. The resulting scores plot (FIG. 2(a)) presented as a scatter plot and box and whisker plot demonstrated clear differentiation between the RUPP and normal pregnancy profiles with an R²=0.69, Q²=0.63, and an AUC of 0.995. The QC samples were not used in the model construction. These samples were simply projected through the model post-hoc. The relative lack of dispersion of the projected QC samples provided robust quality assurance of the model's precision. Permutation testing showed that the probability of a model of this quality randomly occurring was less than 0.01 (FIG. 7)

FIG. 3. 895 metabolite features were consistently detected in both the cord plasma and RUPP experiments. This bi-plot compares the significance values for these common metabolite features with respect to the cord plasma study (SGA vs Control) and RUPP study (Normal vs. RUPP). Each point in the bi-plot represents one of the observed common metabolite features. A circle indicates a metabolite which significantly changes in both the venous cord plasma and RUPP significance tests. The triangles indicate metabolites that are significantly changed in RUPP but not significantly changed in venous cord plasma, and the squares indicate metabolites that are significantly changed in venous cord plasma but not significantly changed in RUPP. The crosses indicate no significant change in either the SGA or control samples. Points lying in zone A show a mean increase in metabolite level for RUPP samples and a mean decrease in venous cord plasma samples; zone B show a mean increase in metabolite level for both venous cord plasma and RUPP samples; zone C show a decrease in mean metabolite level for both venous cord plasma and RUPP samples; zone D show a decrease in mean metabolite level for RUPP samples and an increase for venous cord plasma samples.

FIG. 4. 785 metabolite features were consistently detected in both the venous cord plasma and week-15 experiments. The bi-plot compares the univariate significance values for these common metabolite features. Each point in the bi-plot represents one of the observed common metabolite features with respect to the venous cord plasma study (SGA vs Control) and week-15 study (SGA vs Control). A circle indicates a metabolite which significantly changes in both the venous cord plasma and week-15 significance tests. The triangles indicate metabolites that are significantly changed in week-15 but not significantly changed in venous cord plasma, and the squares indicate metabolites that are significantly changed in venous cord plasma but not significantly changed in week-15. The crosses indicate no significant change in either the venous cord plasma or week-15 samples. Points lying in zone A show a mean increase in metabolite level for week-15 samples and a mean decrease in venous cord plasma samples; zone B show a mean increase in metabolite level for both venous cord plasma and week-15 samples; zone C show a decrease in mean metabolite level for both venous cord plasma and week-15 samples; zone D show a decrease in mean metabolite level for week-15 samples and an increase for venous cord plasma samples.

FIGS. 5A-5C. The PLS-DA model predictions for the final 19-metabolite signature found by the Genetic Algorithm Search program. FIG. 5A depicts model predictions for the week-15 plasma data. R²=0.61, Q²=0.56, an AUC of 0.90 and an optimal odds ratios of 44 (95% CI 9-214). FIG. 5B depicts model predictions for the venous cord plasma data. R²=0.83, Q²=0.81, and an AUC of 1. FIG. 5C demonstrates that of 19 signature metabolites 11 were detected in the RUPP model analysis. The PLS-DA model built using these metabolites gave an R²=0.66, Q²=0.65, and an AUC of 0.98. Permutation testing showed that the probability of models of this quality randomly occurring is less than 0.001 in all cases (FIG. 9).

FIG. 6. A cross-validated PLS-DA model of all the venous cord plasma metabolite features detected was built using two latent factors with an R²=0.88, Q²=0.81, and an AUC of 1. Here a reference Q² distribution is obtained by calculating all possible PLS-DA models under random reassignment of the case/control labels for each measured metabolic profile. If the correctly labeled model's R2 (vertical line) value is close to the centre of the reference distribution then the model performs no better than a randomly assigned model and is therefore invalid. A non-parametric test comparing the ‘candidate’ model (vertical line) and the permuted H₀ distribution (histogram) showed that the probability of a model of this quality randomly occurring was less than 0.001.

FIG. 7. A cross-validated PLS-DA model of all the RUPP plasma metabolite features detected was built using 2 latent factors with an R²=0.69, Q²=0.63, and an AUC of 0.995. A non-parametric test comparing the ‘candidate’ model (vertical line) and the permuted H₀ distribution (histogram) showed that the probability of a model of this quality randomly occurring was less than 0.01.

FIGS. 8A-8B. FIG. 8A depicts graphs of a cross-validated PLS-DA model (3 latent variables) constructed using the week-15 data, using only those metabolites that previously showed significant difference in the venous cord plasma study and were reproducibly detected in the week-15 study (n=516). This had a Q²=0.48, R²=0.43, AUC of 0.94 and an optimal discriminatory odds ratio of 49 (95% CI 13-184). FIG. 8B depicts a non-parametric test comparing the ‘candidate’ model (red line) and the permuted H₀ distribution (blue histogram) showed that the probability of a model of this quality randomly occurring was less than 0.05.

FIGS. 9A-9B. The PLS-DA model predictions for the final 19-metabolite signature found by the Genetic Algorithm Search program. FIG. 9A depicts model predictions for the week-15 plasma data. R²=0.61, Q²=0.56, an AUC of 0.90 and an optimal odds ratios of 44 (95% CI 9-214). A non-parametric test comparing the ‘candidate’ model (vertical line) and the permuted H₀ distribution (histogram) showed that the probability of a model of this quality randomly occurring was less than 0.001. FIG. 9B depicts model predictions for the venous cord plasma data. R²=0.83, Q²=0.81, and an AUC of 1. A non-parametric test comparing the ‘candidate’ model (vertical line) and the permuted H₀ distribution (histogram) showed that the probability of a model of this quality randomly occurring was less than 0.001. FIG. 9C depicts model predictions for the RUPP data. R²=0.66, Q²=0.65, and an AUC of 0.98. A non-parametric test comparing the ‘candidate’ model (vertical line) and the permuted H₀ distribution (histogram) showed that the probability of a model of this quality randomly occurring was less than 0.001. In the Detailed Description that follows, the numbers in bold face type serve to identify the component parts that are described and referred to in relation to the drawings depicting various embodiments of the invention. It should be noted that in describing various embodiments of the present invention, the same reference numerals have been used to identify the same of similar elements. Moreover, for the sake of simplicity, parts have been omitted from some figures of the drawings.

### DETAILED DESCRIPTION

As will be described in more detail below, in one aspect, the present invention relates to compounds, compositions and methods for the use of metabolites to produce a metabolic profile of a disorder or disease in a subject, and the analysis of such a metabolic profile(s) in order to identify disturbances in such profiles in a subject which are caused by or correlated with the diseases or disorders. In one example, the disease or disorder is SGA and/or IUGR.

In another aspect, the present invention relates to compounds, compositions and methods for the use of a metabolic profile of a disorder or disease in a subject, to provide an indication of the risk of pregnancy associated disorder or disease in a subject. In one example, the disease or disorder is SGA and/or IUGR.

In yet another aspect the present invention relates to compounds, compositions and methods for the use of a metabolic profile of a disorder or disease in a subject, to provide an indication of the risk of pregnancy associated disorder or disease in a subject, and allow medical intervention for the benefit of the subject and/or newborn or fetus. Additionally or alternatively, a subject identified to be at risk can be monitored to that appropriate steps or treatment can be taken. In one example, the disease or disorder is SGA and/or IUGR.

In one example, the compositions and methods described herein relate to the detection and/or monitoring of the progression of pregnancy, as well as complications of pregnancy.

The term “progression of pregnancy” as used herein refers to the various stages or phases of pregnancy. The “progression of pregnancy” includes the course of pregnancy in both normal pregnancies and pregnancies in which a complication develops. In one example, the methods, compounds and compositions as described herein are useful to detect and/or aid in the detection of pregnancy complications or risk of developing pregnancy complications such as intrauterine growth restriction (IUGR). In another example, the methods, compounds and compositions as described herein are useful to detect and/or aid in the detection of pregnancy complications or risk of developing pregnancy complications such as small for gestational age infants (SGA).

In a specific example, the compounds, compositions and methods described herein relate to the use of metabolites to produce a metabolic profile and the identification of biomarkers to detect and/or aid in the detection of IUGR.

In a specific example, the compounds, compositions and methods described herein relate to the use of metabolites to produce a metabolic profile and the identification of biomarkers to detect and/or aid in the detection of SGA.

In one example of the methods described herein, a biological sample from a subject is assessed for presence of metabolites within the biological sample, wherein the levels and/or concentration of the metabolites indicates a diagnosis of IUGR or SGA. As used herein, the term “biological sample” or “sample”, and the like, refer to a material known to or suspected of containing or expressing the endogenous metabolite(s) in the profile. The sample can be used directly as obtained from the subject or used following a pre-treatment to modify the character of the biological sample. For example, the biological sample can be treated prior to use, such as preparing plasma from blood, diluting viscous fluids, and the like. Non-limiting methods of treatment of the biological sample include, but are not limited to, filtration, distillation, extraction, concentration, inactivation of interfering components, the addition of reagents, and the like. A biological sample can be derived from any biological source, such as tissues or extracts, including cells, and physiological fluids, such as, for example, whole blood, plasma including venous umbilical cord plasma, serum, saliva, ocular lens fluid, cerebrospinal fluid, sweat, urine, milk, ascitic fluid, synovial fluid, peritoneal fluid and the like.

In a specific example, the biological sample is a biological fluid, more specifically venous cord plasma or peripheral plasma.

As used here, the term “subject” refers a mammal. In a specific example, the subject is a female mammal. In another specific example, the female mammal is a human, In another specific example, the subject is a pregnant female human at about 15 weeks' gestation. I another example, the subject is a pregnant female human at more than about 15 weeks' gestation. In another example, the subject is a pregnant female human at less than about 15 weeks' gestation.

In another example, the female mammal is rat or mouse. In some examples, the subject is a companion animal (dog, cat, and the like) or livestock (cow, horse, and the like).

In one example, the methods described herein comprise the step of obtaining a biological sample directly from the subject and/or directly from one or more controls.

As used herein, “obtaining” a biological sample refers to the methods obtaining a biological sample. Such methods of “obtaining” a biological sample will be well know to the skilled worker. For example, a blood sample may be obtained by venepuncture, as is well known. A biological sample may be obtained directly or indirectly from the subject. The term “obtaining” a biological sample may comprise receiving a biological sample from an agent acting on behalf of the subject. For example, receiving a biological sample from a doctor, nurse, hospital, medical centre, etc., either directly or indirectly, e.g. via a courier or postal service. In some cases the biological sample is obtained from archival repositories. In one example, the methods of the invention are carried out in vitro or ex vivo.

As used herein, the term “control” relates to an individual or group of individuals of the gender and same species as the subject being tested. Examples of characteristics of the controls include, but are not limited to, age, ethnicity, body mass index, systolic blood pressure, diastolic blood pressure, a smoker or non-smoker, gestational stage, combinations thereof, and the like. The “control” will generally be a group of one or more individuals who do not have a disease or disorder as defined herein, and whom do not develop the disease or disorder. Levels for control samples from healthy subjects may be established by prospective and/or retrospective statistical studies. Healthy subjects who have no clinically evident disease or abnormalities may be selected for statistical studies. Diagnosis may be made by a finding of statistically different levels of metabolite profile compared to a control sample or previous levels quantified for the same subject. Accordingly, in one example, the term “control” refers to a pregnant female who does not have and is not at risk of developing a complication of pregnancy, including SGA and/or IUGR.

In one example, metabolite levels in the control may, for example, be available from published charts, computer databases, look-up tables, etc. In other examples, the metabolite levels encompass a level which has previously been determined. Thus the method of the invention is not limited to methods which comprise the step of physically testing the level of endogenous metabolite obtained from a control.

The term “metabolite” as used herein refers to at least one molecule of a specific metabolite up to a plurality of molecules of the said specific metabolite. It is to be understood further that a group of metabolites means a plurality of chemically different molecules wherein for each metabolite at least one molecule up to a plurality of molecules may be present.

A metabolite refers to all classes of organic or inorganic chemical compounds including those being comprised by biological sample. In one example a metabolite is a small molecule compound. In another example, there are a plurality of metabolites. Such a plurality of metabolites representing a metabolome.

Metabolites are typically small molecule compounds, such as substrates for enzymes of metabolic pathways, intermediates of such pathways or the products obtained by a metabolic pathway. Metabolic pathways are well known in the art and may vary between species. Examples of metabolic pathways include the citric acid cycle, respiratory chain, photosynthesis, photorespiration, glycolysis, gluconeogenesis, hexose mono phosphate pathway, oxidative pentose phosphate pathway, production and oxidation of fatty acids, urea cycle, amino acid biosynthesis pathways, protein degradation path ways such as proteasomal degradation, amino acid degrading pathways, biosynthesis or degradation of: lipids, polyketides (such as flavonoids and isoflavonoids), isoprenoids (such as terpenes, sterols, steroids, carotenoids, xanthophylls), carbohydrates, phenylpropanoids and derivatives, alcaloids, benzenoids, indoles, indole-sulfur compounds, porphyrines, anthocyans, hormones, vitamins, cofactors such as prosthetic groups or electron carriers, lignin, glucosinolates, purines, pyrimidines, nucleosides, nucleotides and related molecules such as tRNAs, microRNAs (miRNA) or mRNAs. Examples of metabolites include peptides, oligopeptides, polypeptides, oligonucleotides, polynucleotides and lipids.

Small molecule compound metabolites may be composed of compounds including: alcohols, alkanes, alkenes, alkines, aromatic compounds, ketones, aldehydes, carboxylic acids, esters, amines, imines, amides, cyanides, amino acids, peptides, thiols, thioesters, phosphate esters, sulfate esters, thioethers, sulfoxides, ethers, or combinations or derivatives thereof.

Small molecules may be primary metabolites and/or secondary metabolites. Metabolites may further encompass artificial small molecule compounds. Such artificial small molecule compounds are derived from exogenously provided small molecules which are administered or taken up by an organism but are not primary or secondary metabolites. For instance, artificial small molecule compounds may be metabolic products obtained from drugs by metabolic path ways of the animal.

The term “metabolome” as used herein, refer to plurality of metabolites being comprised by a biological system, such as a cell, tissue, biological fluid or organism, under specific conditions. A metabolome may be represented as a data set that includes concentrations of metabolites in the biological system. In specific example, the biological system is the biological sample obtained. In another specific example, the metabolome is from a biological system which is venous cord plasma or peripheral plasma.

The term “metabolite fingerprint” as used herein refers to a distinct or identifiable pattern of metabolite levels, or ratios of such levels. A metabolite fingerprint may refer to relative levels of metabolites or absolute metabolite concentrations. The metabolite fingerprint can be linked to a tissue, cell type, biological fluid, or to any distinct or identifiable condition that influences metabolite levels (e.g., concentrations) in a predictable or associatable way. The metabolite fingerprint includes the relative as well as absolute levels of specific metabolites.

The complex process of fetal growth is governed by maternal, paternal, fetal and placental factors. Particular interest lies in placental insufficiency as a contributory cause. This is most likely due to a poorly perfused placenta and/or poor placental transport of nutrients associated with reduced placental vascular development in early pregnancy such that the fetus does not receive the necessary nutrients and oxygen needed for optimum growth and development (Gagnon, 2003; Jackson et al., 1995; Kingdom et al., 2000; Trudinger & Giles, 1996). In support of this, placentae from women who deliver SGA infants may have macroscopic evidence of infarction and microscopic changes including increased formation of syncytial knots, reduced cytotrophoblast proliferation and increased apoptosis when compared with placentae from pregnancies resulting in normal birthweight infants (Chen et al., 2002; Smith et al., 1997). Trophoblast differentiation and invasion begin in early pregnancy. We therefore hypothesised that altered levels of associated circulating factors would be detectable in the maternal circulation in early pregnancy prior to the clinical detection of the condition.

Metabolic profiling (Allen et al., 2003a; Dunn et al., 2010; Harrigan & Goodacre, 2003; Kell et al., 2005; O'Hagan et al., 2005), is a powerful systems biology strategy for investigating the low molecular weight biochemicals (metabolites) present in the metabolome of a cell, tissue or organism (Dunn, 2008; Dunn et al., 2005; Kell et al., 2005; Kell & Oliver, 2004). Its position as the final downstream product of gene expression enables the provision of a high resolution multi-factorial phenotypic signature of disease etiology, manifestation or pathophysiology (Dunn et al., 2007; Kell, 2007; Sreekumar et al., 2009; Westerhoff & Palsson, 2004).

Metabolomic technology can be used to analyse many different types of biofluid. Human blood is a complex sample type which generates thousands of metabolites and reflects the metabolism of multiple tissue and cell types in the mammalian body. It has been demonstrated that this technology produces reproducible, robust and valid results in metabolic profiling studies when using blood as an analyte (Dunn et al., 2008b; Zelena et al., 2009). Results of a metabolomic screen on plasma from women with established pre-eclampsia (Kenny et al., 2008; Kenny et al., 2010; Kenny et al., 2005; Turner et al., 2008) have previously been reported.

As described herein, in one example, a metabolomic approach for characterizing the metabolic fingerprint of SGA was undertaken.

Three consecutive and independent studies are described herein, as follows: (i) the time-of-disease metabolic signature of SGA in human plasma from the umbilical cord that drains the placental vasculature was characterized, (ii) the time-of-disease metabolite biomarker signature was compared to the metabolic profile of an animal model of placental insufficiency, the reduced uterine perfusion pressure (RUPP) rat, and (iii) the cord plasma biomarker signature was compared to that from peripheral blood samples collected at 15±1 weeks' gestation from women who subsequently delivered SGA baby, and matched control.

In the case of characterizing the time-of-disease metabolic signature of SGA in human plasma from the umbilical cord that drains the placental vasculature, sampling venous umbilical cord plasma within 20 minutes of birth was undertaken thereby allowing to get both as close as possible to the phenotypic disease endpoint, and gain access to the cause of SGA, i.e. the fetal side of the dysfunctional placenta.

In the case of comparing the time-of-disease metabolite biomarker signature to the metabolic profile of an animal model of placental insufficiency, the reduced uterine perfusion pressure (RUPP) rat, the phenotype of the pups and placentas at birth is comparable to severe growth restriction (Alexander, 2003; Walsh et al., 2009). Several animal models of abnormal fetal growth exist (Anthony et al., 2003; Ergaz et al., 2005), however, most involve either stressing the animal (e.g. hypoxia) or imposing strict dietary regimes. Models of this nature are difficult to interpret using metabolomic technology when peripheral plasma is the analyte. For example, in experiments on placental explants under different oxygen conditions, the change in oxygen tension caused a greater disruption in metabolism than did the difference between disease and controls (Dunn et al., 2009; Horgan et al., 2010). The changes in metabolism due to genotype, diet and/or environment are difficult to differentiate from potential biomarkers due to placental insufficiency. The RUPP model, which involves a mechanical intervention to restrict blood flow to the placenta, removes these concerns as any resulting changes in metabolism is more likely to reflect placental etiology. By comparing the cord plasma metabolome to that of the RUPP model, it was possible to assess to what degree the cord plasma biomarker signature reflected the phenotype of a highly constrained model of placental insufficiency.

In the case of comparing cord plasma biomarker signature to that from peripheral blood samples collected at 15±1 weeks' gestation from women who subsequently delivered a SGA baby, and matched controls, a nested case-control experiment was performed using a subset of women who were participants in the multi-national SCreening fOr Pregnancy Endpoints (SCOPE) study (www.scopestudy.net), a prospective cohort study of healthy nulliparous women. The SCOPE biobank samples are well curated, accompanied by comprehensive metadata, and were proportionally matched to avoid potential sources of bias.

Additionally, comparable metabolite data from both the venous cord blood study and the week-15 study were mined in order to find a simple (n<20), yet robust, metabolite rule that effectively predicts SGA in early pregnancy. The components of this simple metabolite signature were compared across all three studies.

A variety of analytical techniques may be used in measuring metabolites, and generating a metabolic fingerprint of a sample and/or a control. Typically, principal analytical techniques employed include liquid chromatography-coupled tandem mass spectrometry (LCMS and LC-MS/MS), ultra-high performance liquid chromatography-coupled mass spectrometry (UPLC-MS), gas chromatography coupled mass spectrometry (GCMS) and nuclear magnetic resonance spectroscopy (NMR). In the examples as described herein, UPLC-MS is used to measure metabolites in a sample.

As described herein, the metabolic fingerprint of the metabolites within the sample identified subjects at risk of developing SGA and/or IUGR. A metabolic fingerprint of 19 metabolites, shown in Table IV, representing the latent systems-wide interaction in the metabolome was sufficient to produce a robust predictive model of pre-symptomatic SGA with AUC of 0.9. The efficacy of these 19 metabolites was also seen in the venous cord plasma (AUC=1) and, for those detected (11 of the 19) in the RUPP model (AUC=0.98).

The methods as described herein, therefore, provide a method to aid in the prognosis, detection and/or diagnosis in a subject at risk of SGA and/or IUGR, based upon the metabolic fingerprint identified.

In a specific example, the metabolic fingerprint comprises the 19 metabolites shown in Table IV.

In another example, the metabolic fingerprint comprises at least 2, 3, 4, 5, 6, 7, 8, 9, 10, 11, 12, 13, 14, 15, 16, 17, 18 or 19 of the metabolites shown in Table IV.

A standard metabolite profile can be used for comparison to the metabolic fingerprint of a pregnant woman and/or new born to be assessed for risk of developing a complication of pregnancy, such as SGA and/or IUGR. For example, by comparing the level(s) of one or more metabolites in the peripheral plasma of a pregnant woman, or the cord plasma of a new born, to be assessed for risk of developing SGA and/or IUGR to the level(s) of the corresponding the metabolites in the metabolite standard profile, one can determine if there are differences between the two profiles. The metabolite standard/control profile may be preestablished or established by assessing samples run concurrently metabolite levels in a normal control.

In some embodiments, the differences between the two biomarker profiles are significant differences. As used herein, the term “significant difference” is well within the knowledge of a skilled artisan and can be determined empirically with reference to each particular biomarker or panel of biomarkers. For example, a significant difference in the level of a biomarker in a subject at risk of developing SGA or IUGR as compared to a healthy subject (one not at risk of developing SGA and/or IUGR) is any difference in serum level that is statistically significant.

There is shown herein that a metabolic fingerprint that is specific SGA and/or IUGR, and can be established in a subject from a biological sample. In one example, the biological sample is venous cord plasma. In another example, the biological sample is peripheral plasma.

In a specific example, the metabolic profile corresponds to the metabolic profile of Table IV.

In a specific example, the metabolic profile comprises the 19 metabolites shown in Table IV.

In another example, the metabolic profile comprises at least 2, 3, 4, 5, 6, 7, 8, 9, 10, 11, 12, 13, 14, 15, 16, 17, 18 or 19 of the metabolites shown in Table IV.

In accordance with one aspect of the present invention there is provided a method of determining a metabolic fingerprint of SGA and/or IUGR comprising: obtaining a sample from said subject; measuring the metabolites within said sample and generating a metabolic profile of said sample; comparing the metabolic profile of said sample with a control metabolic profile, wherein said metabolic fingerprint is determined from the comparison of said sample and said control metabolic profiles.

In practice, the metabolite fingerprint of a biological sample may be determined using any suitable means, as would be know to the skilled worker. It will be appreciated that one or more suitable means may be used to measure metabolites with a biological sample. In one example, the metabolites measured are those in Table IV.

Measurement of a metabolite may be performed by a direct or indirect detected means.

For example, the metabolite fingerprint may be measure using one or more of the analytic techniques described above. In one example, metabolite levels can be measured by one or more method(s) selected from spectroscopy methods such as NMR (nuclear magnetic resonance), or mass spectroscopy (MS); SELDI (-TOF), MALDI (-TOF), a 1-D gel-based analysis, a 2-D gel-based analysis, liquid chromatography (e.g. UPLC-MS, high pressure liquid chromatography (HPLC) or low pressure liquid chromatography (LPLC)), thin-layer chromatography, and LC-MS-based techniques.

The metabolites may be detected directly, or indirectly, via interaction with a ligand or ligands, such as an enzyme, binding receptor or transporter protein, peptide, aptamer, or oligonucleotide, or any synthetic chemical receptor or compound capable of specifically binding the metabolite. The ligand may possess a detectable label, such as a luminescent, fluorescent or radioactive label, and/or an affinity tag. Immunological methods may also be used to detect metabolites within a sample. Lipids and their derivates may also be detected using methods known to the skilled worker. For example, lipids may be extracted using a fluid extractant comprising a non-polar component and a polar component. The individual lipids may then be identified as would be known to the skilled worker. Additional suitable methods include electrochemical, fluorimetric, luminometric, spectrophotometric, polarimetric, chromatographic or similar techniques.

In accordance with one aspect of the present invention, there is provided a method for determining SGA and/or IUGR in a subject, said method comprising analyzing a sample from said subject for a metabolic fingerprint of SGA and/or IUGR.

In accordance with one aspect of the present invention, there is provided a method comprising: obtaining a sample from a subject with, or suspected as having, SGA and/or IUGR; contacting the sample with a reagent to metabolite from the metabolic fingerprint for SGA and/or IUGR the reagent and the metabolite present in the sample; c) measuring the complex formed to determine an amount the metabolite in the sample, wherein the determination of SGA and/or IUGR is determined by the level of the metabolite in said sample.

In accordance with another aspect of the present invention, there is provided a method comprising: obtaining a biologic sample from a subject with, or suspected as having, SGA and/or IUGR; analyzing the sample using a machine wherein said machine having a detector set to detect metabolites within said complex to obtain metabolic fingerprint of the sample; determining SGA and/or IURG in said subject, wherein the determination of SGA and/or IURG in the subject is determined by the levels of the metabolites in said sample.

In another aspect of the present invention, the profile of metabolites may also be used as a tool for screening and identification of a compound(s) and/or composition(s) which act to restore normal levels of the metabolites from a sample from a subject with SGA and/or IUGR, thereby preventing or delaying SGA and/or IUGR, and thus being efficacious in the treatment of SGA and/or IUGR.

As used herein, the term “compound” refers to any chemical entity, pharmaceutical, drug, and the like that can be used to treat or prevent a disease, illness, condition, or disorder of bodily function. A compound can be determined to be therapeutic by screening using the methods of the present invention. Examples of test compounds include, but are not limited to, peptides, polypeptides, synthetic organic molecules, naturally occurring organic molecules, nucleic acid molecules, and combinations thereof.

In another aspect of the present invention, the methods described herein may be carried out using a diagnostic kit for determining the metabolite profile in a biological sample. Such a kit preferably contains regents to identify the metabolites of the metabolic profile and instructions for the use thereof. In a specific example, the kit contains reagent to identify the metabolic profile of Table V. In a specific example, the kit further comprises at least one control sample.

In another aspect of the present invention there is provided a kit for determining the risk of a subject developing a pregnancy with SGA and/or IUGR comprising: instructions for determining a metabolic fingerprint in a biological sample from a subject; a reagent(s) for measuring the metabolic fingerprint in the biological sample from the subject, wherein the determination of SGA and/or IUGR in a subject indicated by the metabolite in the metabolic fingerprint in the sample. In one example control samples are also include. In one example, positive and/or negative control samples are also included in the kit.

In another aspect of the present invention, there is provided herein methods for the treatment of SGA and/or IUGR in a subject. The treatment of SGA and/or IUGR involves reducing, preventing or delaying the symptoms of SGA and/or IUGR in a fetus that already has SGA and/or IUGR. The prevention of SGA and/or IUGR involves reducing, preventing or delaying SGA and/or IUGR in a fetus that does not have SGA and/or IUGR but is at risk of developing the condition. The conditions of fetuses at risk of developing IUGR or displaying the symptoms of IUGR can therefore be improved by administration of a substance used in the inhibition or prevention of IUGR. A therapeutically effective amount of a substance used in the inhibition or prevention of the development of IUGR is preferably given to the mother of the fetus. A determination that a pregnancy is at risk of SGA and/or IUGR enables clinical intervention to manage the course of the disease and/or avoid a poor outcome.

Examples of clinical intervention include, but are not limited to: (i) Stratification of antenatal care—increased visits for those at risk with a corollary reduction in the current number of visits for those not at risk, (ii) for those at risk—increased surveillance including serial ultrasound assessment of fetal growth and wellbeing, (iii) administration of existing agents known to increase fetal growth (limited efficacy)—such as low dose aspirin, (iv) entry into trial of novel agents (which appear promising) such as PDE5 inhibitors (eg Viagra) or (v) consideration of expedition of delivery by induction of labour/Caesarean section if fetal compromise (growth or wellbeing) identified, and the like.

The various methods described herein can be implemented in part or in whole using computer-based systems and methods. Additionally, computer-based systems and methods can be used to augment or enhance the functionality described above, increase the speed at which the functions can be performed, and provide additional features and aspects as a part of or in addition to those of the invention described elsewhere in this document.

A processor-based system can include a main memory, preferably random access memory (RAM), and can also include a secondary memory. The secondary memory can include, for example, a hard disk drive and/or a storage drive (e.g., a removable storage drive), representing a floppy disk drive, a magnetic tape drive, an optical disk drive, etc. The storage drive reads from and/or writes to a machine-readable (computer-readable) storage medium, which refers to a floppy disk, magnetic tape, optical disk, and the like, which is read by and written to by a storage drive. As will be appreciated, the machine-readable storage medium can comprise computer software and/or data, e.g., in the form of tables, databases, or spreadsheets.

The secondary memory may include other similar means for allowing computer programs or other instructions to be loaded into a computer system. Such means can include, for example, a storage unit and an interface. Examples of such can include a program cartridge and cartridge interface (such as the found in video game devices), a movable memory chip (such as an EPROM or PROM) and associated socket, and other storage units (e.g., removable storage units) and interfaces, which allow software and data to be transferred from the storage unit to the computer system.

The computer system can also include a communications interface. Communications interfaces allow software and data to be transferred between computer system and external devices. Examples of communications interfaces can include a modem, a network interface (such as, for example, an Ethernet card), a communications port, a PCMCIA slot and card, and the like. Software and data transferred via a communications interface are in the form of signals, which can be electronic, electromagnetic, optical, or other signals capable of being received by a communications interface. These signals are provided to communications interface via a channel capable of carrying signals and can be implemented using a wireless medium, wire or cable, fiber optics, or other communications medium. Some examples of a channel can include a phone line, a cellular phone link, an RF link, a network interface, and other communications channels.

Computer programs (also referred to computer control logic) are stored in main memory and/or secondary memory. Computer programs can also be received via a communications interface. Such computer programs, when executed, enable the computer system to perform the features of the methods described herein. In particular, the computer programs, when executed, enable the processor to perform the features or steps of the new methods. Accordingly, such computer programs represent controllers of the computer system.

In an embodiment where the elements are implemented using software, the software may be stored in, or transmitted via, a computer-readable medium and loaded into a computer system using a removable storage drive, hard drive or communications interface. The control logic (software), when executed by the processor, causes the processor to perform the functions of the methods described herein.

In another embodiment, the elements are implemented primarily in hardware using, for example, hardware components such as PALs, application specific integrated circuits (ASICs), or other hardware components. Implementation of a hardware state machine so as to perform the functions described herein will be apparent to persons skilled in the relevant art(s). In yet another embodiment, elements are implemented using a combination of both hardware and software.

To gain a better understanding of the invention described herein, the following examples are set forth. It should be understood that these examples are for illustrative purposes only. Therefore, they should not limit the scope of this invention in anyway.

### EXAMPLES

### Example

### Materials and Methods—I

### Participants and Specimens

### (a) Venous Cord Plasma

Venous cord blood was obtained, within 20 minutes of delivery with written maternal consent in compliance with the Central Manchester Research Ethics Committee approval. Blood was collected from women with uncomplicated, term pregnancies resulting in delivery of a healthy singleton fetus (n=6) and from women with suspected SGA, which was subsequently confirmed after delivery on individualised birthweight centiles (n=8) (Gardosi et al., 1992) (www.gestation.net). Pregnancies complicated by any other maternal or fetal factor, including pre-eclampsia, gestational hypertension, diabetes mellitus and congenital anomalies were excluded.

Plasma samples (3 replicates per subject) were collected into BD EDTA-Vacutainer® tubes, placed on ice and centrifuged at 2400 g at 4° C. for 10 minutes according to a standardised protocol. Plasma was stored in aliquots at −80° C. The collection and storage conditions were identical for cases and controls.

### (a) RUPP Model

Pregnant Sprague Dawley rats (12 weeks; supplied and maintained by the Biological Services Unit, University College Cork) were housed in the Biological Services Unit at University College Cork. Animals were maintained at a temperature of 21±2° C., with a 12-hour light/dark cycle and with free access to food and tap water. All procedures were performed in accordance with national guidelines and the European Community Directive 86/609/EC and approved by the University College Cork Local Animal Experimentation Ethics Committee.

On day 14 of a 21 day pregnancy, animals destined for the RUPP experimental group were anesthetized with isoflurane (2% to 5% inhalation) and the abdominal cavity opened via a midline incision to expose the lower abdominal aorta. A silver clip (0.203 mm ID) was placed around the aorta (above the iliac bifurcation) to reduce uterine perfusion pressure by approximately 40% (Eder & McDonald, 1988). Because compensation of blood flow to the placenta occurs via an adaptive response of the uterine arteries (Nienartowicz et al., 1989), silver clips (0.10 mm ID) were also placed on the main uterine branches of both right and left uterine arteries. A series of experiments was also carried out in sham-operated animals (i.e. subjected to the same surgical procedure with the exception that the vessels were not partially occluded). On day 19 of pregnancy, all animals were anesthetized with isoflurane and blood was collected via the abdominal aorta into pre-cooled heparinised vacutainers. All pups and placentas were removed, weighed, and litter size noted. Any animals in which the clipping procedure had resulted in total reabsorption of fetuses were excluded from the study. Blood collected into pre-cooled heparinised vacutainers was centrifuged at 2400 g for 10 minutes at 4° C.; the plasma was then removed and stored in 250 μL aliquots at −80° C. Plasma for a total of 23 animals was collected for metabolomic analysis: 7 normal pregnant, 8 sham operated and 8 RUPP.

### (c) Week-15 Peripheral Plasma

All women were participants in the multi-national SCreening fOr Pregnancy Endpoints (SCOPE) study (www.scopestudy.net). These samples are extremely well curated, accompanied by comprehensive metadata, and are proportionally population matched to avoid potential sources of bias (Broadhurst & Kell, 2006). The SCOPE study is a prospective, cohort study with the main aim of developing accurate screening methods for later pregnancy complications, including SGA (ACTRN12607000551493). Full ethical approval has been obtained and all patients gave written informed consent. Healthy nulliparous women with a singleton pregnancy were recruited between 14 and 16 weeks' gestation and tracked throughout pregnancy

We performed a case control study within the initial 596 recruits from Adelaide, Australia, of whom pregnancy outcome was known in 595 (99.8%). Seventy-three (12.2%) women went on to deliver SGA babies and 267 (44.8%) had uncomplicated pregnancies. The remainder had other pregnancy complications. Forty women who developed SGA were matched for age, ethnicity and BMI to 40 controls who had uncomplicated pregnancies. Women with co-existent pre-eclampsia were excluded from the study.

Venipuncture was performed at 15±1 weeks' gestation, and plasma samples were collected into BD EDTA-Vacutainer® tubes, placed on ice and centrifuged at 2400 g at 4° C. according to a standardised protocol. Plasma was stored in aliquots at −80° C. The collection and storage conditions were identical for cases and controls, with the time between collection and storage being 2.07 (SD 0.90) and 2.02 (SD 0.96) hours, respectively P=0.78.

### Reagents, Sample Preparation and Mass Spectral Analysis

All chemicals and reagents used were of Analytical Reagent or HPLC grade and purchased from Sigma-Aldrich (Poole, UK) or ThermoFisher Scientific (Loughborough, UK). Plasma samples were allowed to thaw on ice for 3 hours, vortex mixed to provide a homogeneous sample and deproteinised. To 100 μl of plasma was added 300 μl methanol (HPLC grade) followed by vortex mixing (15 seconds, full speed) and centrifugation (15 minutes, 11 337 g). 270 μl aliquots of the supernatant were transferred to a 2 ml tube and lyophilised (HETO VR MAXI vacuum centrifuge attached to a Thermo Svart RVT 4104 refrigerated vapor trap; Thermo Life Sciences, Basingstoke, U.K.). Quality Control (QC) samples were obtained by pooling 50 μl aliquots from each plasma sample prepared. This was defined as the pooled QC sample and 100 μl aliquots were deproteinised as described herein.

Deproteinised samples were prepared for UPLC-MS analysis by reconstitution in 90 μl HPLC grade water followed by vortex mixing (15 seconds), centrifugation (11 337 g, 15 minutes) and transfer to vials. Samples were analysed by an Acquity UPLC (Waters Corp. Milford, USA) coupled to a hybrid LTQ-Orbitrap mass spectrometry system (Thermo Fisher Scientific, Bremen, Germany) operating in electrospray ionisation mode as previously described (Dunn et al., 2008a; Zelena et al., 2009). Samples were analyzed in batches of up to 120 samples, with an instrument maintenance step at the end of each batch involving mass spectrometer ion source and liquid chromatography column cleaning. For each analytical batch a number of pooled QC samples were included to provide quality assurance. The first ten injections were pooled QC samples (to equilibrate the analytical system) and then every 5th injection was a pooled QC sample. For each of the analytical experiments (venous cord plasma/RUPP/week-15) sample preparation order was randomised from sample picking and re-randomised before sample analysis in order to ensure no systematic biases (e.g. analysis order correlates with sample preparation order). The samples were also blinded to the analytical scientists to avoid any subjective bias. Each study was performed several months apart, such that all the studies could be considered independent both in terms of sample source and chemical analysis. Raw profile data was deconvolved into a peak table using XCMS software (Brown et al., 2009). Data was then subjected to strict Quality Assurance procedures so that statistical analysis was only performed on reproducible data. Full details of all methods pertaining to sample preparation and UPLC-MS analysis, and quality assurance are described in the attached supplementary methodology file.

### Statistical Analysis

Comparisons of clinical data between cases and controls were performed using the Student's t-test, Mann—Whitney test, Chi square test or Fisher's Exact test, as appropriate (SAS® system 9.1).

For each metabolite peak reproducibly detected in a given study, the null hypothesis that the means of the case and control sample populations were equal was tested using either the Mann-Whitney test or Student's t-test, depending on data normality (assessed using the Lilliefors test). The critical p-value for significance was set to 0.05. Avoiding false positives by correcting for multiple comparisons was performed using False Discovery Rate (FDR) (Storey, 2002) analysis and FDRs are quoted where appropriate. Comparisons across experiments were not corrected as this process of validation is deemed sufficient to remove any false positives. In addition, a Receiver-Operator Characteristic (ROC) curve was calculated to assess each peak's effectiveness as a univariate discriminatory biomarker. The area under the ROC curve (AUC) provides a good estimate of biomarker utility (an AUC=1 demonstrates perfect biomarker separation; AUC=0.5 demonstrates no utility at all).

Multivariate profile-wide predictive models were constructed using Partial Least Squares Discriminant Analysis (PLS-DA) (Eriksson et al., 2001; Wold, 1975; Wold et al., 2001). For each model all the reproducible peaks for a given study were included, unless expressly stated. The number of latent variables in each model was selected using stratified 5-fold cross validation (Eriksson et al., 2001), and associated R², Q², and calculated. Where R², the squared correlation coefficient between the dependant variable and the PLS-DA prediction, measures ‘goodness-of-fit’ (a value between zero and one, where one is a perfect correlation) using all the available data to build a given PLS-DA model. Q² provides a measure of ‘goodness-of-prediction’, and is the averaged correlation coefficient between the dependent variable and the PLS-DA predictions for the 5-hold out data sets generated during the cross-validation process.

Further validation was performed to check the robustness of the final PLS-DA model by comparing it's Q² value to a reference distribution of all possible models using permutation testing (N=1000) following the standard protocol for metabolomic studies (Westerhuis et al., 2008). Here a reference Q² distribution is obtained by calculating all possible PLS-DA models under random reassignment of the case/control labels for each measured metabolic profile. If the correctly labeled model's Q² value is close to the centre of the reference distribution then the model performs no better than a randomly assigned model and is therefore invalid. For all PLS-DA models described here the associated reference distribution plots are provided, from which an estimate of the probability of the candidate model randomly occurring can be estimated. In addition, for each PLS-DA model, a ROC curve was determined so that an accurate assessment of discriminatory ability could be made.

Finally, we searched for an ‘optimal’ multivariate discriminatory model drawn from the named metabolites observed in both the venous cord plasma and week-15 studies. A Genetic Algorithm-based search program was used to obtain the subset of metabolites which produced an effective predictive rule for the onset of SGA. This search method has been shown to be very successful in previous studies (Allen et al., 2003b; Broadhurst et al., 1997; Cavill et al., 2009; Goodacre & Kell, 2003; Jarvis & Goodacre, 2005; Kell, 2002). In this algorithm, a set of candidate solutions evolve over time toward an optimal state. The evolution is pushed by computational techniques inspired by evolutionary biology. In our algorithm, each candidate solution (subset of metabolites) is assessed by building two independent Linear Discriminant Analysis models, one modeling the venous cord plasma data, and the other modeling the week-15 data. A candidate's fitness is proportional to the sum of the root mean square error of prediction (RMSEP) of these two models. Once the optimal subset of metabolites was found, its predictive ability was assessed using PLS-DA. Assessment was performed independently for the venous cord plasma and week-15 data. In addition the final ‘rule’ was tested using the RUPP model data to see if there was a consistent minimal signature across all three studies.

All peak data were Pareto scaled before multivariate analysis (van den Berg et al., 2006). All statistical analyses were carried out using the Matlab® scripting language (http://www.mathworks.com/). All univariate algorithms were implemented such that any missing values are ignored. All multivariate algorithms were implemented such that missing values were imputed using the nearest-neighbor method (Speed, 2003).

Where appropriate, for the PLS-DA prediction scores, the optimal unbiased discriminatory decision boundary was estimated using the optimal Youden's index method (Youden, 1950) and then the associated discriminatory odds ratios with 95% confidence intervals (OR 95% CI) calculated (Perkins & Schisterman, 2006; Youden, 1950).

### Metabolite Identification

For identification of UPLC-MS peaks, the accurate mass for each peak was searched against The Manchester Metabolomics Database (Brown et al., 2009) constructed with information from metabolic reconstructions (Herrgard et al., 2008), both HMDB (http://www.hmdb.ca/) and Lipidmaps (http://www.lipidmaps.org/). A metabolite name(s) was reported when a match with a mass difference between observed and theoretical mass was less than 3 ppm. Using UPLC-MS, metabolites are often detected multiple times due to chemical adduction, dimerization, multiple-charging, isotope peaks and fragmentation. After removal of duplicate identifications, a list of unique metabolites was compiled. Definitive identifications were reported only for metabolites with retention time errors<10 seconds and an accurate mass match<5 ppm. Once identified, the metabolites were grouped into metabolite classes using the HMDB ‘Class’ hierarchy (http://www.hmdb.ca/).

### Materials and Methods—II

### UPLC-MS Analysis

Samples were prepared by reconstitution in 70 μl HPLC grade water followed by vortex mixing (15 seconds), centrifugation (11 337 g, 15 minutes) and transfer to vials. Samples were analysed by an Acquity UPLC (Waters Corp. Milford, USA) coupled to a LTQ-Orbitrap mass spectrometry system (Thermo Fisher Scientific, Bremen, Germany) operating in electrospray ionisation mode. Samples were analysed consecutively in positive ion mode followed and then consecutively in negative ion mode. Chromatographic separations were performed employing an ACQUITY UPLC BEH 1.7 μm-C₁₈ column (2.1×100 mm, Waters Corp. Milford, USA). Solvent A and solvent B were 0.1% formic acid in water and 0.1% formic acid in methanol, respectively. In positive ion mode a flow rate of 0.40 ml·min⁻¹ was applied with a gradient elution profile (100% A for 1 minute and subsequently ramped to 100% B (curve 5) over 15 minutes, followed by a 4 minute hold at 100% B before a rapid return to 100% A and a hold for 2 minutes). In negative ion mode a flow rate of 0.36 ml·min⁻¹ was applied with a gradient elution program (100% A for 2 minutes and subsequently ramped to 100% B (curve 4) over 15 minutes, followed by a 5 minute hold at 100% B before a rapid return to 100% A and a hold for 2 minutes). The column and samples were maintained at temperatures of 50° C. and 4° C., respectively. A 10 μl sample volume was introduced onto the column and 50% of the column effluent was transferred to the mass spectrometer. Centroid MS scans were acquired in the mass range of 50-1000Th using the Orbitrap mass analyser operating with a target mass resolution of 30 000 (FWHM as defined at m/z 400) and a scan time of 0.4 s. Mass calibration was performed before each analytical batch using an instrument manufacturer defined calibration mixture (ThermoFisher Scientific, Bremen, Germany).

### Data Processing of UPLC-MS Data

All data was converted to netCDF format using the FileConverter program in the XCalibur software package (ThermoFisher Scientific, Bremen, Germany). Raw data processing. All raw data (in raw file format) were converted to netCDF file format with the FileConverter program available in XCalibur (ThermoFisher Scientific, Bremen, Germany).

### XCMS Deconvolution

XCMS is an open-source deconvolution program available for LC-MS data. (Smith, C. A. et al. 2006) Deconvolution using the XCMS program was performed using identical settings to those reported previously (Brown, M. et al. 2009) with the exception of s/n threshold=3, step=0.02, m/z diff=0.05 and for grouping bandwidth=10 and mzwidth=0.05. The esi program (http://msbi.ipb-halle.de/msbi/esi/) available with the XCMS software package was used to write peak output files to an annotated version (as a .csv file) which is more appropriate for these studies. XCMS and esi were run using R version 2.6.0.

### Quality Assurance

The performance of analytical instrumentation has to be assessed robustly to ensure that data are of comparable high quality within an analytical run. An approach based on the periodic analysis of a standard biological Quality Control sample (QC sample) together with the patient samples is now accepted as a quality assurance strategy in metabolic profiling. (van der Greef, J et al. 2007; Zelena, E. et al. 2009) A similar Quality Assurance protocol has been followed in this metabolomic study to assess the repeatability for thousands of endogenous metabolites. A set of pooled QC samples were prepared by mixing equal aliquots from all the samples in a single study. A QC sample is then injected after every fourth patient sample in each analytical run (a lead-in of 10 consecutive QC injections was performed at the start of every analytical run to equilibrise the IPLC column response). At the end of the experimental run, and after XCMS deconvolution each detected peak is normalised to the QC sample using robust Loess signal correction (R-LSC). Here Locally Weighted Scatterplot Smoothing (LOESS) is performed on the QC data with respect to the order of injection. A cubic spline correction curve for the whole analytical run is then interpolated, to which the total data set for that peak is normalized. Using this procedure any attenuation of peak response over an analytical run (i.e. confounding factor due to injection order) is minimised. (van der Greef, J et al. 2007; Zelena, E. et al. 2009) After R-LSC each peak is required to pass strict Quality Assurance criteria. While there are no generally accepted criteria for the assessment of repeatability in metabolomic data sets, the UK Food and Drug Administration (FDA) suggests a range of criteria that should be applied. In the guidance for bioanalytical method validation in industry (CDER. 2001) the FDA recommends for single analyte tests that tolerance limits are set such that the measured response detected in two-thirds of QC samples is within 15% of the QC mean, except for compounds with concentrations at or near the limit of quantification (LOQ), in these cases a tolerance of 20% is acceptable. In our case, the methods are not specific for one analyte of interest, but instead we aim to detect thousands of analytes, therefore an acceptance tolerance of 20% would seem to be appropriate. Any peak that did not pass the QA criteria was removed from the dataset and thus ignored in any subsequent data analysis.

### Results

### Venous Cord Plasma

Maternal characteristics and pregnancy outcomes of the cases and controls are described in Table I. Age, BMI, parity, smoking and baby sex were carefully matched across cases and controls. All SGA babies had an individualised birthweight centile<10^{th} centile.

TABLE I
Maternal characteristics and pregnancy outcomes of the SGA and
control babies from which the venous cord plasma samples were taken.

| Maternal | Control | SGA | |
|---|---|---|---|
| Characteristics | n = 6 | n = 8 | p-value |
| Age (years) | 24.5 (20.8-31.2) | 30 (26.3-32.5) | 0.33 |
| Nullip | 4 | 5 | 1.00 |
| BMI Kg/m² | 25.0 (22.0-27.1) | 24.8 (21.8-25.5) | 0.82 |
| Current smoker | 1 | 2 | 1.00 |
| Sex (male) | 4 | 4 | 0.63 |
| Mode of Delivery | | | |
| vaginal | 4 | 2 | 0.28 |
| C/S | 2 | 6 | |
| Gestation at | 38.5 (38.2-38.7) | 39.0 (38.2-40.0) | 0.15 |
| delivery (weeks) | | | |
| Ethnicity | | | |
| caucasian | 4 | 3 | 0.59 |
| other | 2 | 5 | |
| Birthweight (g) | 3040 (2945-3240) | 2735 (2419-2813) | 0.05 |
| Customized | 29 (19-58) | 3 (2-5) | 0.04 |
| birthweight centile | | | |

Values are median (interquartile range) or number.
BMI = body mass index;
C/S = caesarean section.

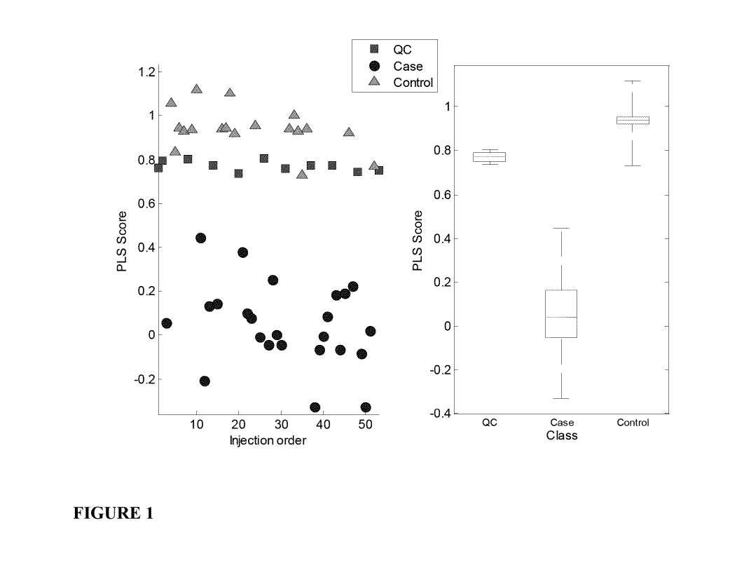

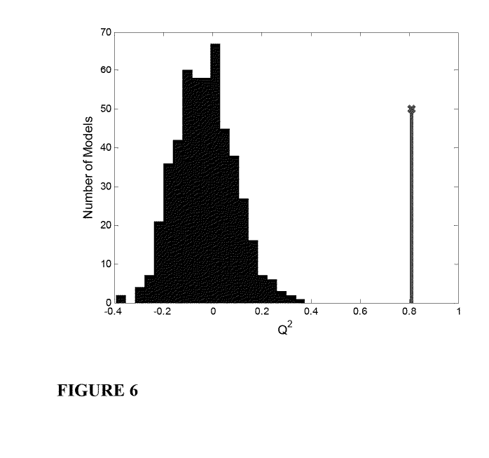

Ultra Performance Liquid Chromatography-Mass Spectrometry (UPLC-MS) analysis reproducibly detected a total of 2011 metabolite features. A cross-validated Partial Least Squares Discriminant Analysis (PLS-DA) model was built using two latent factors. The resulting scores plot (FIG. 1) demonstrated clear differences between the SGA and control profiles with an R²=0.88, Q²=0.81, and an AUC of 1. Permutation testing showed that the probability of a model of this quality randomly occurring was less than 0.001 (FIG. 6)

Univariate hypothesis testing was performed across the 2011 detected metabolites. Prior to statistical analysis, the three analytical replicates were averaged, to avoid biased reduction of the significance values. With a critical p-value of 0.05, 744 metabolite features (37% of those detected) were found to have significant difference between SGA and control, with a false discovery rate (FDR) of 6%, of which 96 were putatively identified as ‘unique’ endogenous metabolites.

### RUPP Model

RUPP pups were associated with restricted fetal growth, with respect to pup weight, when compared with normal pregnant (2.2±0.1 versus 3.2±0.1 g; P<0.001) and sham-operated (2.2±0.1 versus 3.2±0.1 g; P<0.001) pups (data not shown). Furthermore, placental weights from RUPP rats were also significantly reduced compared with both normal pregnant (0.33±0.01 versus 0.43±0.01 g; P<0.001) and sham operated (0.33±0.01 versus 0.42±0.02 g; P<0.001) rats (data not shown) (Walsh et al., 2009).

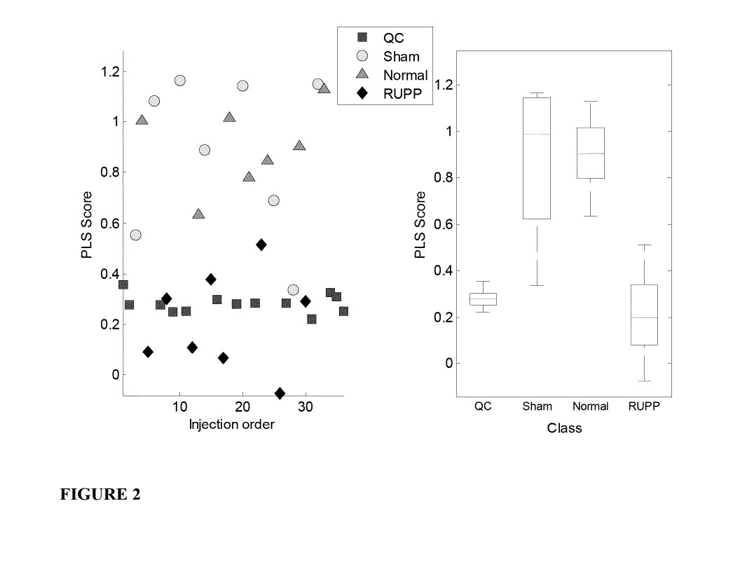

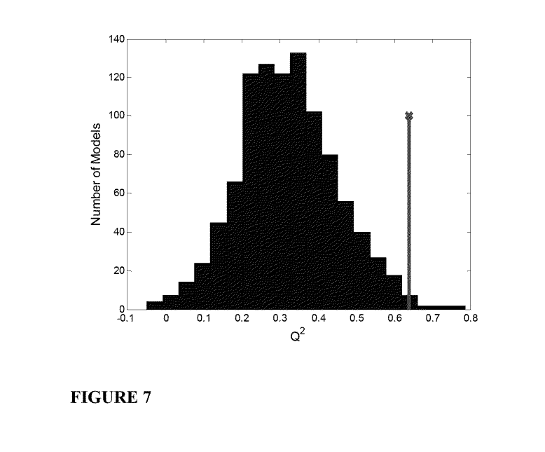

UPLC-MS analysis reproducibly detected a total of 2008 metabolite features. A cross-validated PLS-DA model was built using 2 latent factors. The resulting scores plot (FIG. 2) demonstrated clear differentiation between the RUPP and normal pregnancy profiles with an R²=0.69, Q²=0.63, and an AUC of 0.995. Permutation testing showed that the probability of a model of this quality randomly occurring was less than 0.01 (FIG. 7).

Univariate hypothesis testing was performed across the 2008 detected metabolite features. With a critical p-value of 0.05, 602 metabolite features were found to have significant difference between RUPP vs. normal pregnant/Sham (FDR of 9%), of which, 45 were putatively identified as ‘unique’ endogenous metabolites.

Comparison of Venous Cord Plasma Biomarkers with RUPP Biomarkers

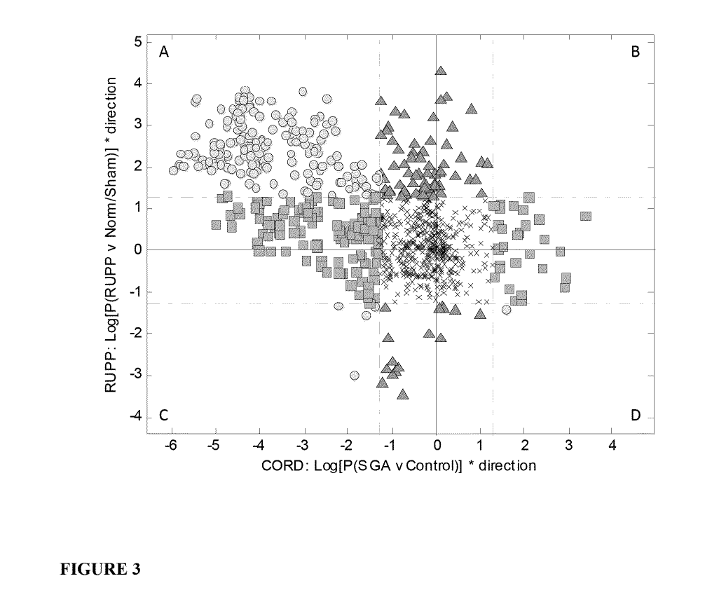

895 metabolite features were consistently detected in both the venous cord plasma and RUPP experiments. FIG. 3 compares the significance values for these common metabolites between the cord plasma study (SGA vs. Control) and RUPP plasma study (normal pregnant vs. RUPP). Each point in the bi-plot represents one of the observed common metabolites. Each point's co-ordinate location (x,y), is defined by the significance of the difference in mean metabolite concentration for the given case/control hypothesis test (x=venous cord plasma; y=RUPP) combined with the mean direction of the difference in metabolite concentration (+ve=case>control; −ye=case<control). 193 metabolite features were significantly different in both studies (22% of the total detected). Ninety-six percent of these metabolite features showed an increase in metabolite level for the cases, with respect to control, in the RUPP model and a decrease for the cases, with respect to control, in the cord plasma. Table SI lists the putatively identified metabolites that were significantly different in both studies.

TABLE SI
The putatively identified metabolites that were significantly different
between the cord plasma study (SGA vs. Control) and RUPP plasma study (Normal vs. RUPP).

| | Venous | | | |
|---|---|---|---|---|
| | Cord Plasma | | RUPP model | |
| Putative Metabolite Inde nty based on exact mass | p-value | direction | p-value | direction |
| Adamantane-1-Carboxylic Acid-5-Dmethylamino-Naphthalene-1-Sulfonylamino-Octyl-Amide_Cervonyl | 3.09E−06 | DOWN | 0.004 | UP |
| carnitine AND/OR 1α,25-dihydroxy-18-oxocholecalciferol | | | | |
| PC(20:4/0:0) AND/OR LysoPC(20:4) | 6.57E−06 | DOWN | 0.001 | UP |
| LysoPC(14:0) OR PC(O-12:0/2:0)ORlysoPC(14:0) OR 1,25-dihydroxy-24-oxo-23-azaergocalciferol | 6.63E−06 | DOWN | 0.008 | UP |
| LysoPC(18:2) | 7.81E−06 | DOWN | 0.012 | UP |
| PC(O-16:1/2:0) AND/OR PC(16:0/2:0) AND/OR PC(18:1/0:0) AND/OR LysoPC(18:1) AND/OR | 8.42E−06 | DOWN | 0.003 | UP |
| LysoPC(20:4) | | | | |
| 1α,25-dihydroxy-24-oxo-23-azaergocalciferol | 9.55E−06 | DOWN | 0.008 | UP |
| LysoPC(16:1) OR Cervonyl carnitine | 1.10E−05 | DOWN | 0.005 | UP |
| LysoPC(18:0) OR PC | 3.80E−05 | DOWN | 0.0004 | UP |
| Clupanodonyl carnitine AND/OR Vaccenyl carnitine | 3.90E−05 | DOWN | 0.002 | UP |
| N-propyl-16,16-dimethyl-5Z,8Z,11Z,14Z-docosatetraenoyl amine | 5.30E−05 | DOWN | 0.025 | UP |
| LysoPC(16:0) OR PC(O-14:0/2:0) OR Docosa-4,7,10,13,16-pentaenoyl carnitine OR Clupanodonyl | 5.50E−05 | DOWN | 0.002 | UP |
| carnitine | | | | |
| Docosa-4,7,10,13,16-pentaenoyl carnitine OR Clupanodonyl carnitine | 6.40E−05 | DOWN | 0.004 | UP |
| 12-oxo-c-LTB3 AND/OR S-(9-deoxy-delta9,12-PGD2)-glutathione | 6.90E−05 | DOWN | 0.014 | UP |
| LysoPC(18:1) OR LysoPC(18:1) OR PC(O-16:1/2:0) | 7.60E−05 | DOWN | 0.008 | UP |
| 4-Amino-N-{4-[2-(2,6-Dimethyl-Phenoxy)-Acetylamino]-3-Hydroxy-1-Isobutyl-5-Phenyl- | 9.40E−05 | DOWN | 0.001 | UP |
| Pentyl}-Benzamide OR 3-Amino-N-{4-[2-(2,6-Dimethyl-Phenoxy)-Acetylamino]-3- | | | | |
| Hydroxy-1-Isobutyl-5-Phenyl-Pentyl}-Benzamide | | | | |
| Taurocholate OR Taurohyocholate OR Tauroursocholic acid OR Taurallocholic acid OR Tauro-b- | 1.32E−04 | DOWN | 0.010 | UP |
| muricholic acid | | | | |
| LysoPC(18:0) OR PC(O-16:0/2:0) OR tetracosapentaenoyl carnitine | 4.35E−04 | DOWN | 0.001 | UP |
| tetracosapentaenoyl carnitine | 4.74E−04 | DOWN | 0.002 | UP |
| Phosphatidate | 0.001 | DOWN | 0.015 | UP |
| LysoPC(18:0) OR PC | 0.001 | DOWN | 0.002 | UP |
| PC OR leukotriene C5 | 0.002 | DOWN | 0.004 | UP |
| PC(O-14:0/18:0) OR PC(O-16:0/16:0) OR Tricosanamide OR | 0.002 | DOWN | 0.027 | UP |
| Linoelaidyl carnitine OR (+/−)N-(1-methyl-2-hydroxy-2-phenyl-ethyl) arachidonyl amine OR N- | 0.002 | DOWN | 0.004 | UP |
| eicosanoyl-ethanolamine | | | | |
| 1-18:1-lysoPE OR PE(18:1(9Z)/0:0) OR PE(18:1(9Z)/0:0)[U] OR N-(5-hydroxy-pentyl) | 0.002 | DOWN | 0.012 | UP |
| arachidonoyl amine OR N-propyl N-(2-hydroxy-ethyl) arachidonoyl amine OR (+)N-(2S-hydroxy-propyl) | | | | |
| a,a-dimethylarachidonoyl amine OR N-(2-isopropyl-5Z,8Z,11Z,14Z-eicosatetraenoyl)-ethanolamine | | | | |
| OR N-(5Z,8Z,11Z,14Z-tricosatetraenoyl)-ethanolamine OR N-(17,17-dimethyl-5Z,8Z,11Z,14Z- | | | | |
| heneicosatetraenoyl)-ethanolamine OR N-(17-methyl-5Z,8Z,11Z,14Z-docosatetraenoyl)- | | | | |
| ethanolamine | | | | |
| N-(11Z-eicosaenoyl)-ethanolamine OR Palmitoylcarnitine OR 1-(1Z-hexadecenyl)-sn-glycero-3- | 0.004 | DOWN | 0.046 | UP |
| phosphoethanolamine OR CPA(18:0/0:0) | | | | |
| PC(O-16:0/18:1) AND/OR PC(O-18:0/16:1) AND/OR PC(16:0/18:0) | 0.004 | DOWN | 0.001 | UP |
| Tocopherol nicotinate OR 11a-(4-dimethylaminophenyl)-1a,25-dihydroxyvitamin D3 OR Tocopherol | 0.005 | DOWN | 0.001 | UP |
| nicotinate OR PC(O-18:0/O-2:1) | | | | |
| Stearoylglycerone phosphate OR 1-Oleoyl-lysophosphatidic acid OR LPA(/18:1) OR lysoPE(16:0) OR | 0.005 | DOWN | 0.005 | UP |
| lysoPC(13:0) OR Aplidiasphingosine OR Sphingofungin A | | | | |
| 1α,25-dihydroxy-11α-phenylcholecalciferol | 0.006 | DOWN | 0.049 | DOWN |
| (15Z)-Tetracosenoic acid OR 10,13-Dimethyl-11-docosyne-10,13-diol OR trans-selacholeic acid OR | 0.006 | DOWN | 0.023 | UP |
| nervonic acid OR Conessine | | | | |
| N-Glycoloylganglioside GM2 OR PC - more than 20 hits | 0.007 | DOWN | 0.021 | UP |
| PC(16:0/22:5) AND/OR PC(18:0/20:5) AND/OR PC(18:1/20:4) AND/OR PC(16:1/22:4) | 0.014 | DOWN | 0.001 | DOWN |
| PGD2-dihydroxypropanylamine OR 15R-PGE2 methyl ester, 15-acetate | 0.020 | DOWN | 0.019 | UP |

Comparison of Venous Cord Plasma Biomarkers with Week-15 Peripheral Biomarkers

Maternal characteristics and pregnancy outcome of the women who subsequently delivered an SGA baby, and controls, for the week-15 peripheral plasma study are shown in Table II.

TABLE II
Characteristics and pregnancy outcome of women who later
developed SGA and controls in the week-15 study.

| | SGA | | Controls | | |
|---|---|---|---|---|---|
| | n = 40 | | n = 40 | | P-Value |
| Maternal Characteristics | | | | | |
| Age (years) | 23.4 | (5.4) | 24.2 | (5.2) | 0.49 |
| Ethnicity | | | | | |
| Caucasian | 39 | (97.5) | 39 | (97.5) | 1.0 |
| Other | 1 | (2.5) | 1 | (2.5) | |
| At 15 weeks gestation | | | | | |
| Body mass index (kg/m²) | 25.0 | (4.5) | 23.8 | (3.8) | 0.21 |
| Systolic blood pressure | 109 | (9) | 107 | (10) | 0.49 |
| (mmHg) | | | | | |
| Diastolic blood pressure | 64 | (8) | 62 | (7) | 0.26 |
| (mmHg) | | | | | |
| Current smoker | 15 | (37.5%) | 6 | (15%) | 0.02 |
| Gestation at blood sampling | 14.9 | (0.7) | 15.0 | (0.7) | 0.87 |
| (wks) | | | | | |
| Pregnancy Outcome | | | | | |
| Systolic blood pressure | 129 | (15) | 121 | (8) | 0.006 |
| (mmHg) | | | | | |
| Diastolic blood pressure | 76 | (9) | 74 | (6) | 0.23 |
| (mmHg) | | | | | |
| Gestational hypertension | 8 | (13%) | — | | |
| Gestation at delivery (wks) | 39.6 | (1.6) | 40.2 | (1.0) | 0.05 |
| Preterm Delivery (<37 wks) | 3 | (7.5%) | — | | 0.24 |
| Birthweight (g) | 2608 | (309) | 3624 | (359) | <0.0001 |
| Customized birthweight | 4 | (2, 6) | 62 | (44, 76) | <0.0001 |
| centile | | | | | |

Values are mean (SD), median (interquartile range) or number (%).

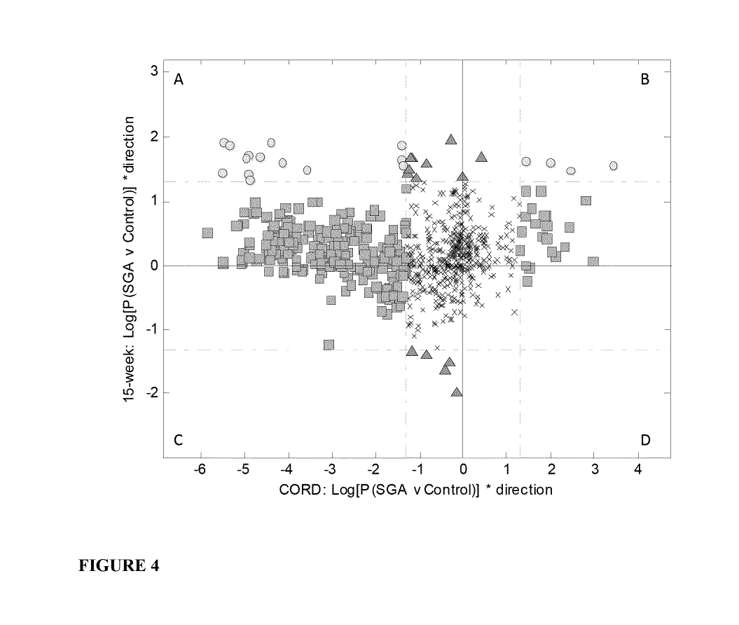

UPLC-MS analysis reproducibly detected a total of 2841 metabolite features. 1202 metabolite features were consistently detected in both the venous cord plasma and week-15 experiments. FIG. 4 compares the univariate significance values for these common metabolite features with respect to the venous cord plasma study (SGA vs Control) and week-15 study (SGA vs Control).

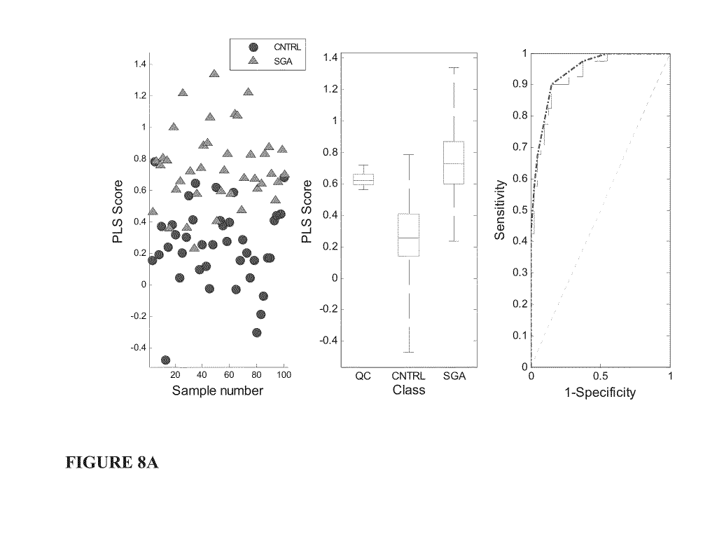

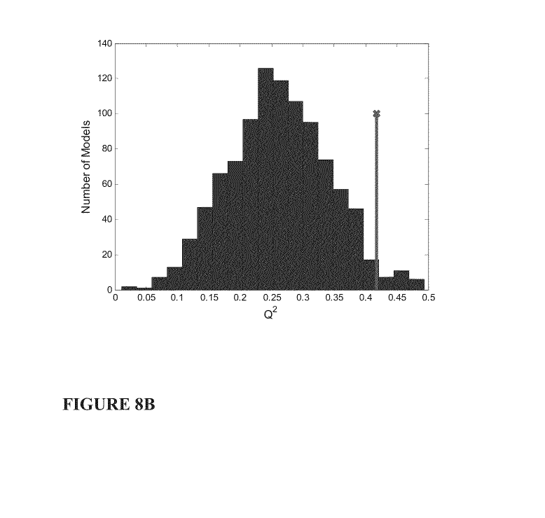

Of the 744 metabolite features that were significant on univariate testing in the venous cord plasma experiment, 516 were also reproducibly detected in maternal peripheral plasma at 15 weeks' gestation. A cross-validated PLS-DA model (3 latent variables) constructed for the week-15 data, using only those 516 candidate SGA biomarkers, produced a Q²=0.48, R²=0.43, AUC of 0.94 and an optimal discriminatory odds ratio of 49 (95% CI 13-184) (FIG. 8a). Permutation testing showed that the probability of a model of this quality randomly occurring was less than 0.02 (FIG. 8b).

Twenty-nine metabolite features were significantly different in both studies (p<0.05), of which 6 were putatively identified as ‘unique’ endogenous metabolites (after removing the multiple matches for chemical adducts and isotope peaks). These are listed in Table III.

TABLE III
The table shows the putatively identified metabolites that were significant
(p < 0.05) in venous cord plasma and the week-15 plasma studies. PC = phospholine,
PGD = Prostaglandin D, PGE = prostaglandin E.

| | Venous | | | |
|---|---|---|---|---|
| | Cord Plasma | | week-15 | |
| Putative Metabolite Indenty based on exact mass | p-value | direction | p-value | direction |
| Pregnanediol-3-glucuronide OR 3alpha,20alpha-dihydroxy- | 0.008 | DOWN | 0.003 | UP |
| 5beta-pregnane 3-glucuronide | | | | |
| LysoPC(16:1) OR Cervonyl carnitine | 1.10E−05 | DOWN | 0.021 | UP |
| 6-hydroxysphingosine OR (4OH,8Z,t18:1) OR 3b- | 0.001 | DOWN | 0.026 | UP |
| Allotetrahydrocortisol OR 15-methyl-15-PGD2 OR 15R-PGE2 | | | | |
| methyl ester | | | | |
| Leucyl-leucyl-norleucine OR Sphingosine 1-phosphate | 0.040 | DOWN | 0.028 | UP |
| Cervonyl carnitine AND/OR 1α,25-dihydroxy-18- | 3.09E−06 | DOWN | 0.035 | UP |
| oxocholecalciferol | | | | |
| 17-[(Benzylamino)methyl]estra-1,3,5(10)-triene-3,17beta-diol | 2.68E−04 | DOWN | 0.045 | UP |

Week-15 peripheral biomarker signature

In order to find a simple, yet robust, predictive algorithm for SGA diagnosis the data from both the venous cord plasma and week-15 studies were mined using a Genetic Algorithm-based search algorithm to find the subset of named metabolites which produced the most robust predictive general model. The Genetic Algorithm chose 19 metabolites (Table IV).

Table IV. The table shows the putatively identified metabolites that were used in the final 19 metabolite predictive venous cord plasma and week-15 plasma models. P-values for those metabolites detected in the RUPP model are included for comparison. DG=Diglyceride, PC=Phosphocholine, PGD=Prostaglandin D, PGE=prostaglandin E.

TABLE IV

| | | Venous Cord | | | | | |
|---|---|---|---|---|---|---|---|
| | | Plasma | | week-15 | | RUPP | |
| Putative Metabolite Indenty based on exact mass | HMDB Class | p-value | direction | p-value | direction | p-value | direction |
| Phenylacetylglutamine OR Formyl-N-acetyl-5- | Amino Acids OR | 0.15 | DOWN | 0.06 | DOWN | | |
| methoxykynurenamine | Amino Ketones | | | | | | |
| Leucyl-leucyl-norleucine OR Sphingosine 1-phosphate | Amino Acids OR | 0.04 | DOWN | 0.03 | UP | 0.19 | UP |
| | Sphingolipids | | | | | | |
| Cervonyl carnitine AND/OR 1α,25-dihydroxy-18- | Carnitines or | 3.09E−06 | DOWN | 0.004 | UP | 0.04 | UP |
| oxocholecalciferol | Vitamin D | | | | | | |
| | derivatives | | | | | | |
| (15Z)-Tetracosenoic acid OR 10,13-Dimethyl-11-docosyne- | Fatty Acids | 0.006 | DOWN | 0.28 | UP | 0.02 | UP |
| 10,13-diol OR trans-selacholeic acid | | | | | | | |
| Hexacosanedioic acid | Fatty Acids | 0.02 | DOWN | 0.19 | UP | | |
| Pentacosenoic acid OR Teasterone OR Typhasterol | Fatty Acids | 0.02 | DOWN | 0.21 | UP | | |
| Cycloheptanecarboxylic acid OR Cyclohexyl acetate | Fatty Acids OR | 0.09 | DOWN | 0.07 | UP | | |
| OR Octenoic acid OR Methyl-heptenoic acid OR 4- | Amino Acids | | | | | | |
| hydroxy-2-octenal OR DL-2-Aminooctanoic acid OR | | | | | | | |
| 3-amino-octanoic acid | | | | | | | |
| DG(14:0/18:0) OR DG(16:0/16:0) | Glycerolipids | 0.01 | UP | 0.39 | UP | 0.09 | UP |
| LysoPC(18:2) | Glycerolipids | 2.27E−06 | DOWN | 0.37 | DOWN | 0.005 | DOWN |
| Hydroxybutyrate OR Hydroxy-methylpropanoate OR Methyl | Hydroxy Acids | 0.08 | DOWN | 0.08 | UP | 0.23 | UP |
| methoxyacetate | | | | | | | |
| LysoPC and PC - more than 10 hits | Phosphocholine | 6.57E−06 | DOWN | 0.25 | UP | 0.001 | UP |
| PC - more than 20 hits | Phosphocholine | 0.08 | DOWN | 0.04 | DOWN | | |
| PC or ubiquinone-8 | Phosphocholine | 0.01 | DOWN | 0.2 | DOWN | 0.66 | DOWN |
| Acetylleucyl-leucyl-norleucinal OR Oleoylglycerone | Phospholipids | | | 0.08 | UP | | |
| phosphate OR LPA(0:0/18:2(9Z,12Z)) OR 1-16:1- | | | | | | | |
| lysoPE OR PC(O-11:1(10E)/2:0) OR (3s)-3,4-Di-N- | | | | | | | |
| Hexanoyloxybutyl-1-Phosphocholine OR N-(3- | | | | | | | |
| hydroxy-propyl) arachidonoyl amine OR N-(2- | | | | | | | |
| methoxy-ethyl) arachidonoyl amine OR N-methyl N- | | | | | | | |
| (2-hydroxy-ethyl) arachidonoyl amine OR SIMILAR | | | | | | | |
| LysoPC(16:1) OR Cervonyl carnitine | Phospholipids OR | 1.10E−05 | DOWN | 0.02 | UP | 0.005 | UP |
| | Carnitines | | | | | | |
| Sphinganine 1-phosphate | Sphingolipids | 0.14 | DOWN | 0.03 | UP | 0.09 | UP |
| Sphingosine 1-phosphate | Sphingolipids | 0.08 | DOWN | 0.05 | UP | 0.58 | UP |
| Pregnanediol-3-glucuronide OR 3alpha,20alpha- | Steroid conjugates | 0.008 | DOWN | 0.003 | DOWN | | |
| dihydroxy-5beta-pregnane 3-glucuronide | | | | | | | |
| 6-hydroxysphingosine OR (4OH,8Z,t18:1) sphingosine | Steroids and | 0.001 | DOWN | 0.02 | UP | | |
| OR 15-methyl-15-PGD2 OR 15R-PGE2 methyl ester | Steroid Derivatives | | | | | | |

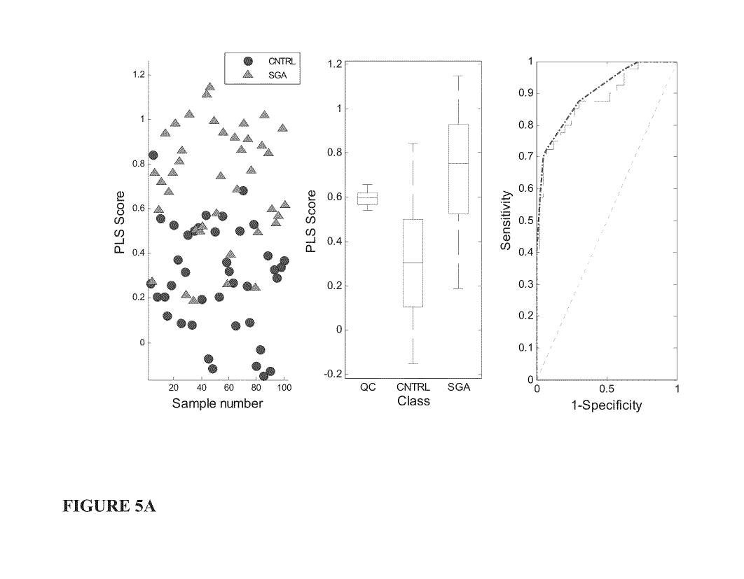

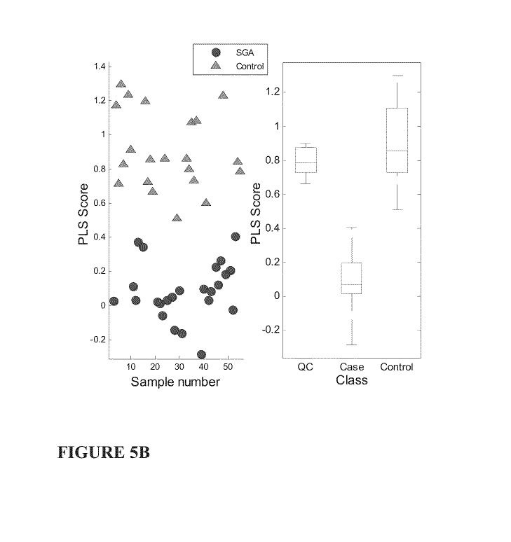

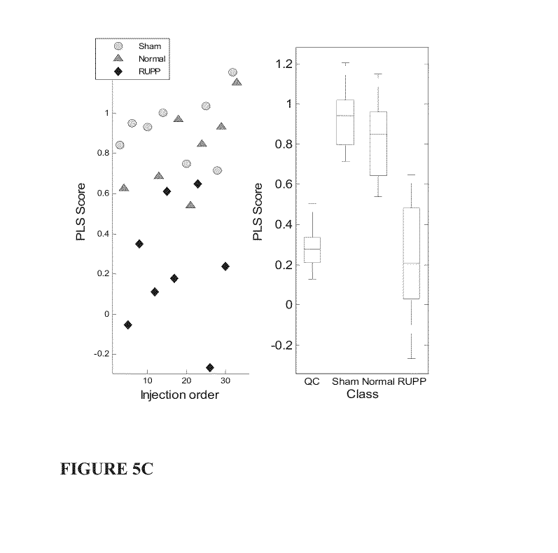

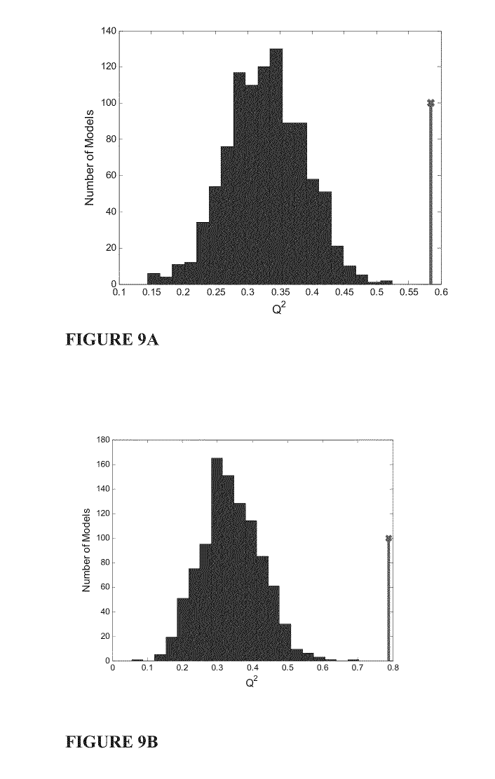

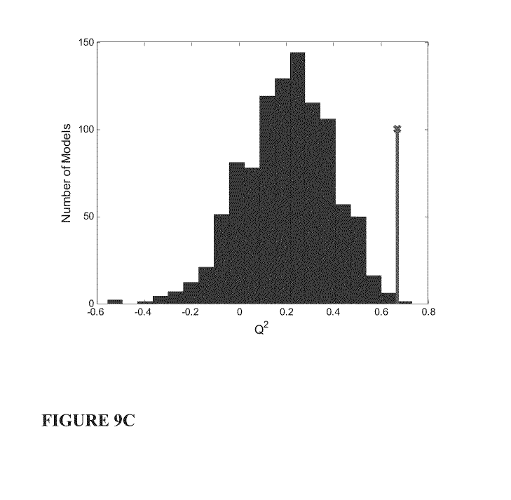

FIG. 5(a&b) shows the PLS-DA model predictions using these metabolites for both the week-15 study and the venous cord plasma study. For the week-15 data, the 19 metabolite model had an R²=0.61, Q²=0.56, an AUC of 0.90 and an optimal odds ratios of 44 (95% CI 9-214). For the venous cord plasma data the 19 metabolite model had an R²=0.83, Q²=0.81, and an AUC of 1. Permutation testing showed that the probability of either of these models randomly occurring was less than 0.001 (Supplementary Figure S4). Of the 19 signature metabolites, 11 were also detected in the RUPP model study. The PLS-DA RUPP model built using only these metabolites gave an R²=0.66, Q²=0.65, and an AUC of 0.98 (FIG. 5c). Again, permutation testing showed that the probability of models of this quality randomly occurring is less than 0.001 (FIG. 9).

### DISCUSSION

Accumulating evidence suggests that small for gestational age is a complex syndrome with multiple biological pathways contributing to the etiology. We have, therefore, taken a holistic and data-driven, systems biology approach (Kell, 2004) to identify a metabolic signature in plasma that is predictive of SGA. The overall study comprised of three consecutive independent studies: (a) time-of-disease biomarker discovery in cord blood plasma originating from the placenta, (b) biomarker validation in an animal model (c) validation of biomarkers in a pre-symptomatic clinical setting.

Using robust data mining and modeling techniques in these three independent studies, it has been shown that a combination of 19 metabolites representing the latent systems-wide interaction in the metabolome is sufficient to produce a robust predictive model of pre-symptomatic SGA with AUC of 0.9 (FIG. 5). The efficacy of these 19 metabolites is also seen in the venous cord plasma (AUC=1) and, for those detected (11 of the 19) in the RUPP model (AUC=0.98). When the metabolites are combined into a single multifactorial model the power of such data-driven technology proves its worth. The need for such a multifactorial approach reflects the high probability that complex diseases such as SGA have more than one cause.

### (a) Time-of-Disease Biomarker Discovery

Metabolic profiling of venous umbilical cord plasma revealed comprehensive disruption of metabolism in SGA babies when compared with normal weight controls. In total, 744 metabolite features (96 putatively identified) were found to be significantly different in the SGA plasma when compared to the normal controls. Multivariate modeling using PLS-DA) confirmed these findings with a predictive sensitivity of 1, and specificity of 1. By assessing SGA at time-of-disease and as close as possible to the hypothesised placental (dys)functional mechanism, evidence of a systemic change in metabolism due to this condition has been uncovered.

### (b) Biomarker Validation in an Animal Model

Comparison of pup birthweight and placental weight in RUPP rats and normal pregnant and sham-operated rats showed that the RUPP rats produced both smaller pups and smaller placentas, characteristics of restricted growth (and therefore SGA). Again, comprehensive disruption of metabolism was observed when comparing the metabolic profiles in plasma of RUPP with normal pregnant rats. When the venous cord plasma biomarker signature was compared to the RUPP biomarker signature there was significant overlap (FIG. 3). From this figure it is also clear that, irrespective of significance, the majority of the detected change in the metabolome appears in quadrant A. This indicates a reduction in metabolite levels due to SGA in cord plasma originating from the placental bed and elevation in systemic metabolite levels due to reduced uterine perfusion pressure in rat plasma. This differential change may well reflect that the sampling locations were on either side of the placental barrier at time-of-disease. The “mirror image” responses may be indicative of the failure of the placenta to regulate the required nutrients and oxygen needed for successful fetal growth and development. Placental dysfunction may reduce the essential metabolites passing through the placental-barrier to the fetus, and the excess dissipated back into maternal blood; thus increasing the detected metabolite levels in maternal SGA plasma.

A number of the putatively identified metabolites in both cord and RUPP plasma demonstrated disruption in carnitine metabolism. These were all decreased in cord and increased in RUPP plasma. Carnitine is an essential factor in fatty acid metabolism in mammals. Its most important known metabolic function is to transport fatty acids into the mitochondria of cells for oxidation (Borum, 1995). The placenta has a high activity of fatty acid oxidation enzymes (Oey et al., 2003) and where defects in long-chain fatty acid oxidation are noted, there is a higher frequency of SGA (Tyni et al., 1998). Previous studies have also found reduced levels of carnitine and acylcarnitines in cord blood of SGA infants (Akisu et al., 2001; Meyburg et al., 2001). Other rat studies of placental insufficiency have reported down-regulated insulin receptor and reduced expression of enzymes involved in fatty acid formation and oxidation as well as altered skeletal muscle mitochondrial lipid metabolism in the growth-restricted pups (Germani et al., 2008; Lane et al., 2001). These metabolic changes may also play a role in the long-term effects associated with SGA.

### (b) Biomarker Validation in a Pre-Symptomatic Clinical Setting

When the biomarker signature of SGA in cord plasma was investigated in peripheral blood collected at 15±1 weeks' gestation, the disruption of metabolism was consistent with the other two studies; however, the change in metabolism was less severe.

Only 29 metabolite features were significant after univariate testing in both the cord plasma and week-15 studies (10 identified—Table III); however, irrespective of significance, FIG. 4 shows that there is a clear trend for metabolites to have reduced levels in the cord plasma and elevated levels in the peripheral maternal plasma. These findings were consistent with the comparison of cord plasma with RUPP plasma (FIG. 3). While not wishing to be bound by theory, this would again suggest that the source of this disruption is at the placental level.

The multivariate predictive PLS-DA model constructed for the 15-week data, using only those metabolite features that were significant after univariate testing in the cord plasma experiment (n=530) revealed that the changing metabolite levels resulted in a model with AUC of 0.94.

Finally, a 19 named metabolomic signature of pre-symptomatic SGA was uncovered using a Genetic-Algorithm search program, utilising both the venous cord plasma and week-15 data. The final panel of metabolites proved effective at discriminating SGA plasma from controls in both the pre-symptomatic week-15 (AUC=0.9) and the venous cord plasma (AUC=1) data. This suggests that the phenotype of SGA is not only multifactorial, (especially so, early in pregnancy), and that metabolomic analysis provides a predictive early screening test for SGA. Table IV lists the panel of 19 metabolites, their associated individual p-values, and the direction of change for all three studies.

A number of sphingolipids were among this panel of metabolites. Sphingolipids are ubiquitous in mammals, playing important roles in signal transmission and cell recognition and are commonly believed to protect the cell surface against harmful environmental factors by forming a mechanically stable and chemically resistant outer leaflet of the plasma membrane lipid bilayer. In particular, sphingosine 1-phosphate (S1P) has been shown to be an important mediator in the signalling cascades involved in apoptosis, proliferation and stress responses (Maceyka et al., 2002; Spiegel & Milstien, 2002). It is also known that growth restriction is associated with increased apoptosis and reduced cytotrophoblast proliferation (Chen et al., 2002; Smith et al., 1997).

Phospholipids also showed significant disruption. Phospholipids are the major lipid constituents of cell membranes. Changes in normal oxygen tensions, which are associated with the pathophysiology of SGA, can cause changes to glycerophospholipids resulting in many different products which have many different proposed biological properties (Fruhwirth et al., 2007). While not wishing to be bound by theory, the phospholipid changes observed in this study are most likely a result of cell membrane damage leading to the subsequent release of phospholipids. However, there is recent evidence of anti-phospholipid antibodies (and complement activation) co-operating in triggering a local inflammatory process, eventually leading to placental thrombosis, hypoxia, and neutrophil infiltration (Tincani et al., 2009).

### SGA and Fetal Growth Restriction

The combination of those metabolites from the 19-metabolite SGA panel that were detected in the RUPP study (11 of the 19 metabolites) were tested for their multifactorial discriminatory power in the RUPP plasma samples. With a PLS-DA AUC of 0.98, it is probable that there is some causal connection between the SGA metabolite signature and the rat model of severe growth restriction. This is reinforced by the high number of overlapping significant metabolites in the comparison of RUPP to venous cord plamsa (FIG. 3; Table SI).

Fetal growth restriction (FGR) is defined as failure of a fetus to achieve its genetically determined potential size. Currently, classifying babies as growth restricted with a high degree of specificity and sensitivity is a complex process, and is difficult to measure in a general clinical setting. It is common practice for SGA to be used as the surrogate endpoint for FGR (Bobrow & Soothill, 1999; RCOG, 2002). However, not all fetuses that are SGA are pathologically growth restricted and, in fact, as many as 30% may be constitutionally small (McCowan et al., 2005). Therefore, we recognize that there are limitations in the use of the birth weight percentile as a surrogate marker of FGR.

It is possible, given the evidence presented here, that we have in fact found a metabolic signature for FGR rather than the more general disease classification SGA. This hypothesis is reinforced by examining the prediction scores of the 19-metabolite PLS-DA model for the week-15 plasma (FIG. 5a). Although, as a general measure of quality an AUC=0.9 is excellent, it can readily be seen from the predictive scatter plot that several SGA samples are misclassified. Of note, the ‘misclassification rate’ of 30% is close to the estimated misclassification rate using SGA as the endpoint for FGR discussed above. One potential limitation of this study is the number of smokers (SGA=15; Controls=6) and the number of SGA women with Gestational Hypertension (n=8) in the week-15 Adelaide study. Population matching was performed as rigorously as possible; however, our nested case-control study was limited by the size of the overall SCOPE prospective cohort, and excluding these participants would have significantly reduced the power of the study. Moreover, one of the main objectives of our work is to develop a screening test that performs robustly in all populations. The predictive ability of the final model (combined with agreement with the venous cord blood and RUPP studies) clearly outweighs any possible confounding influence of the above factors.

In summary, this combined study clearly illustrates the utility of integrating metabolomic analysis of different sample types when investigating diseases/syndromes which are believed to have complex multi-factorial aetiologies. The unambiguous identification of potential biomarkers at time-of-disease, and thus as close to the clinical endpoint as possible, followed by validation using an animal model with known causality, provided the reasoning for their further investigation in maternal blood at an early, and hence clinically useful, time point.

It is believed this is the first time any clear biomarkers for SGA have been discovered using any technology. A pre-symptomatic predictive test at 15±1 weeks' gestation will have a significant impact on clinical care, allowing scarce resources to be concentrated on those at greatest risk.

### REFERENCES

1. Akisu M, Bekler C, Yalaz M, Huseyinov A, Kultursay N (2001) Free carnitine concentrations in cord blood in preterm and full-term infants with intrauterine growth retardation. Pediatr Int 43: 107-108.
2. Alexander B T (2003) Placental insufficiency leads to development of hypertension in growth-restricted offspring. Hypertension 41: 457-462.
3. Allen J, Davey H M, Broadhurst D, Heald J K, Rowland J J, Oliver S G, Kell D B (2003a) High-throughput classification of yeast mutants for functional genomics using metabolic footprinting. Nature Biotechnology 21: 692-696.
4. Allen J, Davey H M, Broadhurst D, Heald J K, Rowland J J, Oliver S G, Kell D B (2003b) High-throughput classification of yeast mutants for functional genomics using metabolic footprinting. Nat Biotechnol 21: 692-696.
5. Anthony R V, Scheaffer A N, Wright C D, Regnault T R (2003) Ruminant models of prenatal growth restriction. Reprod Suppl 61: 183-194.
6. Barker D J P, Eriksson J G, Forsen T, Osmond C (2002) Fetal origins of adult disease: strength of effects and biological basis. International journal of epidemiology 31: 1235-1239.
7. Barker D J P, Gluckman P D, Godfrey K M, Harding J E, Owens J A, Robinson J S (1993) Fetal Nutrition and Cardiovascular-Disease in Adult Life. Lancet 341: 938-941.
8. Bersinger N A, Odegard R A (2004) Second- and third-trimester serum levels of placental proteins in preeclampsia and small-for-gestational age pregnancies. Acta Obstetricia Et Gynecologica Scandinavica 83: 37-45.
9. Bersinger N A, Odegard R A (2005) Serum levels of macrophage colony stimulating, vascular endothelial, and placenta growth factor in relation to later clinical onset of pre-eclampsia and a small-for-gestational age birth. American Journal of Reproductive Immunology 54: 77-83.
10. Bhatia S, Faessen G H, Carland G, Balise R L, Gargosky S E, Druzin M, El-Sayed Y, Wilson D M, Giudice L C (2002) A longitudinal analysis of maternal serum insulin-like growth factor I (IGF-I) and total and nonphosphorylated IGF-binding protein-1 in human pregnancies complicated by intrauterine growth restriction. Journal of Clinical Endocrinology and Metabolism 87: 1864-1870.
11. Bobrow C S, Soothill P W (1999) Fetal growth velocity: a cautionary tale. Lancet 353: 1460-1460.
12. Borum P R (1995) Carnitine in neonatal nutrition. J Child Neurol 10 Suppl 2: S25-31.
13. Brown, M., Dunn, W. B., Dobson, P., Patel, Y., Winder, C. L., Francis-McIntyre, S., Begley, P., Carroll, K., Broadhurst, D., Tseng, A., et al. 2009. Mass spectrometry tools and metabolite-specific databases for molecular identification in metabolomics. Analyst 134:1322-1332.
14. Broadhurst D, Goodacre R, Jones A, Rowland J J, Kell D B (1997) Genetic algorithms as a method for variable selection in multiple linear regression and partial least squares regression, with applications to pyrolysis mass spectrometry. Analytica Chimica Acta 348: 71-86.
15. Broadhurst D I, Kell D B (2006) Statistical strategies for avoiding false discoveries in metabolomics and related experiments. Metabolomics 2: 171-196.
16. Brown M, Dunn W B, Dobson P, Patel Y, Winder C L, Francis-McIntyre S, Begley P, Carroll K, Broadhurst D, Tseng A, Swainston N, Spasic I, Goodacre R, Kell D B (2009) Mass spectrometry tools and metabolite-specific databases for molecular identification in metabolomics. Analyst 134: 1322-1332.
17. Cavill R, Keun H C, Holmes E, Lindon J C, Nicholson J K, Ebbels T M (2009) Genetic algorithms for simultaneous variable and sample selection in metabonomics. Bioinformatics 25: 112-118.
18. CDER. 2001. Guidance for Industry, Bioanalytical Method Validation. F.a.D.A. Centre for Drug Valuation and Research, editor
19. Chang T C, Robson S C, Boys R J, Spencer J A D (1992) Prediction of the Small-for-Gestational-Age Infant—Which Ultrasonic Measurement Is Best. Obstetrics and Gynecology 80: 1030-1038.
20. Chen C P, Bajoria R, Aplin J D (2002) Decreased vascularization and cell proliferation in placentas of intrauterine growth-restricted fetuses with abnormal umbilical artery flow velocity waveforms. American Journal of Obstetrics and Gynecology 187: 764-769.
21. Chiswick M L. Br Med J (Clin Res Ed) 1985; 291 (6499):845-8.
22. Dimond R. CESDi.1: The report. Mod Midwife 1997; 7 (11):20-2.
23. Dugoff L, Hobbins J C, Malone F D, Porter T F, Luthy D, Comstock C H, Hankins G, Berkowitz R L, Merkatz I, Craigo S D, Timor-Tritsch I E, Carr S R, Wolfe H M, Vidaver J, D'Alton M E (2004) First-trimester maternal serum PAPP-A and free-beta subunit human chorionic gonadotropin concentrations and nuchal translucency are associated with obstetric complications: a population-based screening study (the FASTER Trial). Am J Obstet Gynecol 191: 1446-1451.
24. Dugoff L, Hobbins J C, Malone F D, Vidaver J, Sullivan L, Canick J A, Lambert-Messerlian G M, Porter T F, Luthy D A, Comstock C H, Saade G, Eddleman K, Merkatz I R, Craigo S D, Timor-Tritsch I E, Can S R, Wolfe H M, D'Alton M E (2005) Quad screen as a predictor of adverse pregnancy outcome. Obstet Gynecol 106: 260-267.
25. Duley L et al. Cochrane Database Syst Rev 2004 (1):CD004659
26. Dunn W B (2008) Current trends and future requirements for the mass spectrometric investigation of microbial, mammalian and plant metabolomes. Physical biology 5: 11001.
27. Dunn W B, Bailey N J C, Johnson H E (2005) Measuring the metabolome: current analytical technologies. Analyst 130: 606-625.
28. Dunn W B, Broadhurst D, Atherton H J, Goodacre R, Griffin J L (2010) Systems Level Studies of Mammalian Metabolomes: The Roles of Mass Spectrometry and Nuclear Magnetic Resonance Spectroscopy. Chem Soc Rev DOI: 101039/b906712b.
29. Dunn W B, Broadhurst D, Brown M, Baker P N, Redman C W G, Kenny L C, Kell D B (2008a) Metabolic profiling of serum using Ultra Performance Liquid Chromatography and the LTQ-Orbitrap mass spectrometry system. Journal of Chromatography B-Analytical Technologies in the Biomedical and Life Sciences 871: 288-298.
30. Dunn W B, Broadhurst D, Ellis D I, Brown M, Halsall A, O'Hagan S, Spasic I, Tseng A, Kell D B (2008b) A G C-TOF-M S study of the stability of serum and urine metabolomes during the U K Biobank sample collection and preparation protocols. International journal of epidemiology 37 Suppl 1: i23-30.
31. Dunn W B, Broadhurst D I, Deepak S M, Buch M H, McDowell G, Spasic I, Ellis D I, Brooks N, Kell D B, Neyses L (2007) Serum metabolomics reveals many novel metabolic markers of heart failure, including pseudouridine and 2-oxoglutarate. Metabolomics 3: 413-426.
32. Dunn W B, Brown M, Worton S A, Crocker I P, Broadhurst D, Horgan R, Kenny L C, Baker P N, Kell D B, Heazell A E (2009) Changes in the metabolic footprint of placental explant-conditioned culture medium identifies metabolic disturbances related to hypoxia and pre-eclampsia. Placenta 30: 974-980.
33. Eder D J, McDonald M T (1988) A role for brain angiotensin-II in experimental pregnancy-induced hypertension in laboratory rats. Clinical and Experimental Hypertension Part B-Hypertension in Pregnancy 6: 431-451.
34. Ergaz Z, Avgil M, Ornoy A (2005) Intrauterine growth restriction-etiology and consequences: what do we know about the human situation and experimental animal models? Reprod Toxicol 20: 301-322.
35. Eriksson L, Johansson E, Kettaneh-Wold N, Wold S (2001) Multi- and megavariate data analysis: principles and applications. Umetrics Academy, Umeå.
36. Fruhwirth G O, Loidl A, Hermetter A (2007) Oxidized phospholipids: from molecular properties to disease. Biochim Biophys Acta 1772: 718-736.
37. Gardosi J et al. Bmj 2005; 331(7525):1113-7
38. Gagnon R (2003) Placental insufficiency and its consequences. European Journal of
Obstetrics Gynecology and Reproductive Biology 110: S99-S107.
39. Gardosi J, Chang A, Kalyan B, Sahota D, Symonds E M (1992) Customized Antenatal Growth Charts. Lancet 339: 283-287.
40. Gardosi J, Francis A (1999) Controlled trial of fundal height measurement plotted on customised antenatal growth charts. British Journal of Obstetrics and Gynaecology 106: 309-317.
41. Gardosi J, Kady S M, McGeown P, Francis A, Tonks A (2005) Classification of stillbirth by relevant condition at death (ReCoDe): population based cohort study. BMJ (Clinical research ed 331: 1113-1117.
42. Germani D, Puglianiello A, Cianfarani S (2008) Uteroplacental insufficiency down regulates insulin receptor and affects expression of key enzymes of long-chain fatty acid (LCFA) metabolism in skeletal muscle at birth. Cardiovasc Diabetol 7: 14.
43. Gluckman P D, Hanson M A, Cooper C, Thornburg K L (2008) Effect of in utero and early-life conditions on adult health and disease. The New England journal of medicine 359: 61-73.
44. Goodacre R, Kell D B (2003) Evolutionary computation for the interpretation of metabolome data. In Metabolic profiling: its role in biomarker discovery and gene function, Harrigan G G, Goodacre R (eds) pp 239-256. Kluwer Academic Publishers.
45. GRIT study group BJOG 2003; 110 (1):27-32
46. Hall M H, Chng P K, MacGillivray I (1980) Is routine antenatal care worth while? Lancet 2: 78-80.
47. Harrigan G G, Goodacre R (eds) (2003) Metabolic profiling: its role in biomarker discovery and gene function analysis. Kluwer Academic Publishers, Boston.
48. Herrgard M J, Swainston N, Dobson P, Dunn W B, Arga K Y, Arvas M, Bluthgen N, Borger S, Costenoble R, Heinemann M, Hucka M, Le Novere N, Li P, Liebermeister W, Mo M L, Oliveira A P, Petranovic D, Pettifer S, Simeonidis E, Smallbone K, et al. (2008) A consensus yeast metabolic network reconstruction obtained from a community approach to systems biology. Nat Biotechnol 26: 1155-1160.
49. Horgan R P, Broadhurst D I, Dunn W B, Browne M, Heazell A E P, Kell D B, Baker P N, Kenny L C (2010) Changes in the Metabolic Footprint of Placental Explant-Conditioned Medium Cultured in Different Oxygen Tensions from Placentas of Small for Gestational Age and Normal Pregnancies. Placenta (in press).
50. Hospital N M. Annual report. Dublin 1991.
51. Hovatta O et al. Br J Obstet Gynaecol 1983; 90 (8):691-6. Ahlenius I et al. Acta Obstet Gynecol Scand 1995; 74 (2):109-17.
52. Jackson M R, Walsh A J, Morrow R J, Mullen J B M, Lye S J, Ritchie J W (1995) Reduced placental villous tree elaboration in small-for-gestational-age pregnancies—relationship with umbilical artery Doppler wave-forms American Journal of Obstetrics and Gynecology 172: 518-525.
53. Jarvis R M, Goodacre R (2005) Genetic algorithm optimization for pre-processing and variable selection of spectroscopic data. Bioinformatics 21: 860-868.
54. Kean L et al. Journal of Obs and Gynae 1996;16:77-82.
55. Kell D B (2002) Metabolomics and machine learning: explanatory analysis of complex metabolome data using genetic programming to produce simple, robust rules. Molecular biology reports 29: 237-241.
56. Kell D B (2004) Metabolomics and systems biology: making sense of the soup. Current Opinion in Microbiology 7: 296-307.
57. Kell D B (2007) The virtual human: Towards a global systems biology of multiscale, distributed biochemical network models. Iubmb Life 59: 689-695.
58. Kell D B, Brown M, Davey H M, Dunn W B, Spasic I, Oliver S G (2005) Metabolic footprinting and systems biology: the medium is the message. Nat Rev Microbiol 3: 557-565.
59. Kell D B, Oliver S G (2004) Here is the evidence, now what is the hypothesis? The complementary roles of inductive and hypothesis-driven science in the post-genomic era. Bioessays 26: 99-105.
60. Kenny L C, Broadhurst D, Brown M, Dunn W B, Redman C W G, Kill D B, Baker P N (2008) Detection and identification of novel metabolomic biomarkers in preeclampsia. Reproductive Sciences 15: 591-597.
61. Kenny L C, Broadhurst D I, Dunn W B, Brown M, North R A, McCowan L, Roberts C, Cooper G J S, Kell D B, Baker P N (2010) Robust Early Pregnancy Prediction of Later Preeclampsia Using Metabolomic Biomarkers. Hypertension (in press).
62. Kenny L C, Dunn W B, Ellis D I, Myers J, Baker P N, Kell D B, Consortium G (2005) Novel biomarkers for pre-eclampsia detected using metabolomics and machine learning. Metabolomics 1: 227-234.
63. Kingdom J, Huppertz B, Seaward G, Kaufmann P (2000) Development of the placental villous tree and its consequences for fetal growth. European Journal of Obstetrics Gynecology and Reproductive Biology 92: 35-43.
64. Kok J H, den Ouden A L, Verloove-Vanhorick S P, Brand R (1998) Outcome of very preterm small for gestational age infants: the first nine years of life. British Journal of Obstetrics and Gynaecology 105: 162-168.
65. Lane R H, Kelley D E, Ritov V H, Tsirka A E, Gruetzmacher E M (2001) Altered expression and function of mitochondrial beta-oxidation enzymes in juvenile intrauterine-growth-retarded rat skeletal muscle. Pediatric research 50: 83-90.
66. Low J A, Handleyderry M H, Burke S O, Peters R D, Pater E A, Killen H L, Derrick E J (1992) Association of Intrauterine Fetal Growth-Retardation and Learning-Deficits at Age 9 to 11 Years. American Journal of Obstetrics and Gynecology 167: 1499-1505.
67. Lumley J et al. Cochrane Database Syst Rev 2000(2):CD001055.
68. Maceyka M, Payne S G, Milstien S, Spiegel S (2002) Sphingosine kinase, sphingosine-1-phosphate, and apoptosis. Biochim Biophys Acta Mol Cell Biol Lipids 1585: 193-201.
69. McCowan L M, Harding J E, Stewart A W (2005) Customized birthweight centiles predict SGA pregnancies with perinatal morbidity. BJOG 112: 1026-1033.
70. McIntire D D et al. N Engl J Med 1999; 340 (16):1234-8.
71. Morrison I et al. Am J Obstet Gynecol 1985; 152 (8):975-80.
72. Meyburg J, Schulze A, Kohlmueller D, Linderkamp 0, Mayatepek E (2001) Postnatal changes in neonatal acylcarnitine profile. Pediatric research 49: 125-129.
73. Morris R K, Cnossen J S, Langejans M, Robson S C, Kleijnen J, Ter Riet G, Mol B W, van der Post J A, Khan K S (2008) Serum screening with Down's syndrome markers to predict pre-eclampsia and small for gestational age: systematic review and meta-analysis. BMC Pregnancy Childbirth 8: 33.
74. Morrison I, Olsen J (1985) Weight-Specific Stillbirths and Associated Causes of Death—an Analysis of 765 Stillbirths. American Journal of Obstetrics and Gynecology 152: 975-980.
75. Nienartowicz A, Link S, Moll W (1989) Adaptation of the uterine arcade in rats to pregnancy. J Dev Physiol 12: 101-108.
76. O'Hagan S, Dunn W B, Brown M, Knowles J D, Kell D B (2005) Closed-loop, multiobjective optimization of analytical instrumentation: gas chromatography/time-of-flight mass spectrometry of the metabolomes of human serum and of yeast fermentations. Analytical chemistry 77: 290-303.
77. Oey N A, den Boer M E, Ruiter J P, Wanders R J, Duran M, Waterham H R, Boer K, van der Post J A, Wijburg F A (2003) High activity of fatty acid oxidation enzymes in human placenta: implications for fetal-maternal disease. J Inherit Metab Dis 26: 385-392.
78. Perkins N J, Schisterman E F (2006) The inconsistency of “optimal” cutpoints obtained using two criteria based on the receiver operating characteristic curve. American Journal of Epidemiology 163: 670-675.
79. RCOG (2002) The investigation and management of the small-for-gestational-age fetus. Evidence-based Clinical Guideline No. 31. pp 1-16. RCOG Press, London.
80. Richardus et al. J Perinat Med; 1997 (25):13-24.
81. Saenger P, Czernichow P, Hughes I, Reiter E O (2007) Small for gestational age: short stature and beyond. Endocrine reviews 28: 219-251.
82. Sim D et al. Ultrasound Obstet Gynecol 1993; 3 (1):11-7.
83. Smith S C, Baker P N, Symonds E M (1997) Increased placental apoptosis in intrauterine growth restriction. American Journal of Obstetrics and Gynecology 177: 1395-1401.
84. Smith, C. A., Want, E. J., O'Maille, G., Abagyan, R., and Siuzdak, G. 2006. XCMS: processing mass spectrometry data for metabolite profiling using nonlinear peak alignment, matching, and identification. Anal Chem 78:779-787.
85. Speed T P (2003) Statistical analysis of gene expression microarray data. Chapman & Hall/CRC, Boca Raton, Fla.; London.
86. Spiegel S, Milstien S (2002) Sphingosine 1-phosphate, a key cell signaling molecule. J Biol Chem 277: 25851-25854.
87. Sreekumar A, Poisson L M, Rajendiran T M, Khan A P, Cao Q, Yu J, Laxman B, Mehra R, Lonigro R J, Li Y, Nyati M K, Ahsan A, Kalyana-Sundaram S, Han B, Cao X, Byun J, Omenn G S, Ghosh D, Pennathur S, Alexander D C, et al. (2009) Metabolomic profiles delineate potential role for sarcosine in prostate cancer progression. Nature 457: 910-914.
88. Storey J D (2002) A direct approach to false discovery rates. Journal of the Royal Statistical Society Series B-Statistical Methodology 64: 479-498.
89. Taylor R N, Grimwood J, Taylor R S, McMaster M T, Fisher S J, North R A (2003) Longitudinal serum concentrations of placental growth factor: Evidence for abnormal placental angiogenesis in pathologic pregnancies. American Journal of Obstetrics and Gynecology 188: 177-182.
90. Thornton J G et al. Lancet 2004; 364 (9433):513-20
91. Tincani A, Cavazzana I, Ziglioli T, Lojacono A, De Angelis V, Meroni P (2009) Complement Activation and Pregnancy Failure. Clin Rev Allergy Immunol.
92. Tjoa M L, Mulders M A, van Vugt J M, Blankenstein M A, Oudejans C B, van Wijk I J (2003) Plasma hepatocyte growth factor as a marker for small-for-gestational age fetuses. European journal of obstetrics, gynecology, and reproductive biology 110: 20-25.
93. Trudinger B J, Giles W B (1996) Elaboration of stem villous vessels in growth restricted pregnancies with abnormal umbilical artery Doppler waveforms. British Journal of Obstetrics and Gynaecology 103: 487-488.
94. Turner E, Brewster J A, Simpson N A, Walker J J, Fisher J (2008) Aromatic amino acid biomarkers of preeclampsia—a nuclear magnetic resonance investigation. Hypertens Pregnancy 27: 225-235.
95. Tyni T, Ekholm E, Pihko H (1998) Pregnancy complications are frequent in long-chain 3-hydroxyacyl-coenzyme A dehydrogenase deficiency. Am J Obstet Gynecol 178: 603-608.
96. van den Berg R A, Hoefsloot H C, Westerhuis J A, Smilde A K, van der Werf M J (2006) Centering, scaling, and transformations: improving the biological information content of metabolomics data. BMC Genomics 7: 142.
97. van der Greef, J., Martin, S., Juhasz, P., Adourian, A., Plasterer, T., Verheij, E. R., and McBurney, R. N. 2007. The art and practice of systems biology in medicine: mapping patterns of relationships. J Proteome Res 6:1540-1559.
98. Walsh S K, English F A, Johns E J, Kenny L C (2009) Plasma-mediated vascular dysfunction in the reduced uterine perfusion pressure model of preeclampsia: a microvascular characterization. Hypertension 54: 345-351.
99. Westerhoff H V, Palsson B O (2004) The evolution of molecular biology into systems biology. Nature Biotechnology 22: 1249-1252.
100. Westerhuis J A, Hoefsloot H C J, Smit S, Vis D J, Smilde A K, van Velzen E J J, van Duijnhoven J P M, van Dorsten F A (2008) Assessment of PLSDA cross validation. Metabolomics 4: 81-89.
101. Williams R L et al. Obstet Gynecol 1982; 59 (5):624-32
102. Wold H (1975) Soft Modelling by Latent Variables: The Non-Linear Iterative Partial Least Squares (NIPALS) Approach. In Perspectives in Probability and Statistics, Papers in Honour of M S Bartlett, Gani J (ed) pp 117-142. Academic Press, London.
103. Wold S, Trygg J, Berglund A, Antti H (2001) Some recent developments in PLS modeling. Chemometrics and Intelligent Laboratory Systems 58: 131-150.
104. Woman. National Collaborating Centre for Women's and Children's Health. Antenatal Care London: RCOG Press; 2003
105. Youden W J (1950) Index for Rating Diagnostic Tests. Cancer 3: 32-35.
106. Zelena E, Dunn W B, Broadhurst D, Francis-McIntyre S, Carroll K M, Begley P, O'Hagan S, Knowles J D, Halsall A, Wilson I D, Kell D B (2009) Development of a robust and repeatable UPLC-M S method for the long-term metabolomic study of human serum. Analytical chemistry 81: 1357-1364.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed herein is:

1. A method of predicting a SGA (small-for-gestational age) infant in a patient at a pre-symptomatic gestational stage comprising a step of assaying a biological sample from the patient for abundance of a plurality of metabolite biomarkers selected from the 19 metabolite biomarkers of Table IV, correlating the abundance of the plurality of metabolite biomarkers with a metabolite fingerprint of SGA shown in Table IV, and predicting SGA based on the level of correlation between the abundance of the plurality of metabolite biomarkers and the metabolite fingerprint of Table IV.

2. The method of claim 1, in which the SGA is SGA associated with IUGR (Intrauterine growth restriction).

3. The method of claim 1, in which the biological sample is selected from venous cord blood or maternal peripheral blood.

4. The method of claim 1, in which the biological sample is obtained from the patient at week 15 gestational stage+/−2 weeks.

5. The method of claim 1, in which the biological sample is assayed for substantially all of the 19 metabolite biomarkers of Table IV, and in which the levels of the assayed metabolite biomarkers are correlated with the metabolite fingerprint of SGA shown in Table IV, wherein SGA is predicted based on the level of correlation between the levels of the assayed metabolite biomarkers of Table IV and the metabolite fingerprint of Table IV.

6. The method of claim 1, in which the biological sample is assayed for all of the 19 metabolite biomarkers of Table IV, and in which the levels of the assayed metabolite biomarkers are correlated with the metabolite fingerprint of SGA shown in Table IV, wherein SGA is predicted based on the level of correlation between the levels of the assayed metabolite biomarkers of Table IV and the metabolite fingerprint of Table IV.

7. The method of claim 1, for predicting SGA in a patient at week 15 gestational stage+/−2 weeks comprising a step of assaying a venous cord blood or maternal peripheral blood sample from the patient for 19 metabolite biomarkers of Table IV, correlating the levels of the 19 assayed metabolite biomarkers with the metabolite fingerprint of pre-symptomatic SGA shown in Table IV, and predicting SGA based on the level of correlation between the assayed levels of the 19 metabolite biomarkers of Table IV and the metabolite fingerprint of Table IV.

8. A system for performing a method of predicting a SGA infant in a patient, the system comprising:
a determination system for detecting in a biological sample from the patient abundance of a plurality of metabolite biomarkers selected from Table IV;
optionally, a storage system for storing metabolite biomarker abundance data generated by the determination system;
a comparison system for comparing abundance data from the determination system with a metabolite fingerprint of Table IV to provide a quantitative prediction of SGA; and
a display module for displaying the quantitative prediction of SGA.

9. The system of claim 8, in which the determination system comprises a mass spectrometer or liquid chromatography apparatus.

10. The system of claim 8, in which the determination system is adapted for detecting in a biological sample from the patient abundance of substantially all of the 19 metabolite biomarkers of Table IV.

11. The system of claim 8, in which the determination system is adapted for detecting in a biological sample from the patient abundance of all of the 19 metabolite biomarkers of Table IV.

12. The system of claim 8, in which the SGA is SGA associated with IUGR.

13. The system of claim 8, in which the biological sample is venous cord blood or maternal peripheral blood.

14. The method of claim 1, in which the metabolite biomarkers of Table IV are selected from the group consisting of:
phenylacetylglutamine or formyl-N-acetyl-5-methoxykynurenamine;
leucyl-leucyl-norleucine or sphingosine-1-phosphate;
cervonyl carnitine or 1α,25-dihydroxy-18-oxocholecalciferol;
(15Z)-Tetracosenoic acid or 10,13-Dimethyl-11-docosyne-10,13-diol or trans-selacholeic acid;
hexacosanedioic acid;
Pentacosenoic acid or Teasterone or Typhasterol;
Cycloheptanecarboxylic acid or cyclohexyl acetate or octenoic acid or methyl-heptenoic acid or 4-hydroxy-2-octenal or DL-2-aminooctanoic acid or 3-amino-octanoic acid;
Diglyceride(14:0/18:0) or Diglyceride(16:0/16:0);
Lyso-phosphocholine(18:2);
Hydroxybutyrate or hydroxyl-methylpropanoate or methyl methoxyacetate;
Lyso-phosphocholine and phosphocholine;
Phosphocholine;
Phosphocholine or ubiquinone;
Acetylleucyl-leucyl-norleucinal or oleoylglycerone phosphate or LPA(0:0/18:2(9Z,12Z)) or 1-16:1-lyso-prostaglandin E or phosphocholine(0-11:1(10E)/2:0) or (3s)-3,4-Di-N-hexanoyloxybutyl-1-phosphocholine or N-(3-hydroxy-propyl) arachidonoyl amine or N-(2-methoxy-ethyl) arachidonoyl amine or N-methyl N-(2-hydroxy-ethyl) arachidonoyl amine;
Lyso-phosphocholine (16:1) or cervonyl carnitine;
Sphinganine-1-phosphate;
Sphingosine-1-phosphate;
Pregnanediol-3-glucuronide or 3alpha,20alpha-dyhydroxy-5beta-pregnane 3-glucuronide; and
6-hydroxysphingosine or (40H,8Z,t18:1) sphingosine or 15-methyl-15-PGD2 or 15R-PGE2 methyl ester.

15. The system of claim 8, in which the metabolite biomarkers of Table IV are selected from the group consisting of:
phenylacetylglutamine or formyl-N-acetyl-5-methoxykynurenamine;
leucyl-leucyl-norleucine or sphingosine-1-phosphate;
cervonyl carnitine or 1α,25-dihydroxy-18-oxocholecalciferol;
(15Z)-Tetracosenoic acid or 10,13-Dimethyl-11-docosyne-10,13-diol or trans-selacholeic acid;
hexacosanedioic acid;
Pentacosenoic acid or Teasterone or Typhasterol;
Cycloheptanecarboxylic acid or cyclohexyl acetate or octenoic acid or methyl-heptenoic acid or 4-hydroxy-2-octenal or DL-2-aminooctanoic acid or 3-amino-octanoic acid;
Diglyceride(14:0/18:0) or Diglyceride(16:0/16:0);
Lyso-phosphocholine(18:2);
Hydroxybutyrate or hydroxyl-methylpropanoate or methyl methoxyacetate;
Lyso-phosphocholine and phosphocholine;
Phosphocholine;
Phosphocholine or ubiquinone;
Acetylleucyl-leucyl-norleucinal or oleoylglycerone phosphate or LPA(0:0/18:2(9Z,12Z)) or 1-16: 1-lyso-prostaglandin E or phosphocholine(0-11: 1(10E)/2: 0) or (3s)-3,4-Di-N-hexanoyloxybutyl-1-phosphocholine or N-(3-hydroxy-propyl) arachidonoyl amine or N-(2-methoxy-ethyl) arachidonoyl amine or N-methyl N-(2-hydroxy-ethyl) arachidonoyl amine;
Lyso-phosphocholine (16:1) or cervonyl carnitine;
Sphinganine-1-phosphate;
Sphingosine-1-phosphate;
Pregnanediol-3-glucuronide or 3alpha,20alpha-dyhydroxy-5beta-pregnane 3-glucuronide; and
6-hydroxysphingosine or (4OH,8Z,t18:1) sphingosine or 15-methyl-15-PGD2 or 15R-PGE2 methyl ester.
